(19) United States
(12) Patent Application Publication
(10) Pub. No.: US 2025/0041387 A1
(43) Pub. Date: Feb. 6, 2025

(54) POLYPEPTIDE SEQUENCE OF KDM6B AND APPLICATION OF KDM6B IN REGULATING AND CONTROLLING FUNCTION OF MESENCHYMAL STEM CELL

(71) Applicant: BEIJING STOMATOLOGICAL HOSPITAL, CAPITAL MEDICAL UNIVERSITY, Beijing (CN)

(72) Inventors: Zhipeng FAN, Beijing (CN); Chen ZHANG, Beijing (CN); Yu JIANG, Beijing (CN); Lujue LONG, Beijing (CN); Yangyang CAO, Beijing (CN)

(73) Assignee: BEIJING STOMATOLOGICAL HOSPITAL, CAPITAL MEDICAL UNIVERSITY, Beijing (CN)

(21) Appl. No.: 18/713,894

(22) PCT Filed: Nov. 30, 2021
(86) PCT No.: PCT/CN2021/134280

§ 371 Date: May 28, 2024

(51) Int. Cl. A61K 38/44; A61K 38/17; A61K 38/45

(52) U.S. Cl. CPC A61K 38/44; A61K 38/1709; A61K 38/45

(57) ABSTRACT

Disclosed in the present invention is an application of lysine (K)-specific demethylase 6B (KDM6B) in regulating and controlling the function of a mesenchymal stem cell. WDR5 is a co-binding protein for negatively regulating and controlling the functions of KDM6B and the MLL1. WDR5 can form a protein complex with KDM6B to inhibit the function of KDM6B, such that expression and functions of genes are regulated and controlled by regulating and controlling the methylation state of downstream senescence and osteogenesis related genes and gene promoter region histone, and finally the effects of regulating stem cell senescence and differentiation functions and bone/tooth tissue repair and regeneration functions are achieved. For a KDM6B and WDR5 binding region sequence, small-molecule polypeptide is researched, developed, and utilized, and the function of the mesenchymal stem cell is regulated by regulating and controlling the binding of a KDM6B/WDR5 complex.

### CROSS-REFERENCE TO RELATED APPLICATIONS

This application is a U.S. National Stage application under 35 U.S.C. § 371 of International Application No. PCT/CN2021/134280, filed on Nov. 30, 2021, and entitled “POLYPEPTIDE SEQUENCE OF KDM6B AND APPLICATION OF KDM6B IN REGULATING AND CONTROLLING FUNCTION OF MESENCHYMAL STEM CELL”.

### FIELD

The present invention relates to the technical field of biomedicine and in particular to a polypeptide of KDM6B and use thereof in regulating the function of a mesenchymal stem cell.

### BACKGROUND

With the increase in human lifespan and the development of an aging society, population aging has become an increasingly serious social problem in all countries today. Aging, as a physiological phenomenon in the life process, causes neurodegenerative diseases, osteoporosis, tooth loss, cardiovascular diseases, tumors and metabolic diseases, all of which have become major threats to human health. With aging, the human skeleton is gradually degenerating, and the incidence rate of osteoporosis is increasing significantly. Osteoporosis affects not only the vertebrae, hip bones and phalanges, but also the jaw bones. The more severe the osteoporosis throughout the skeletal system, the more significant the loss of bone and mineralized tissue in the jaw bones. Imbalance of the remodeling of jaw bones, resorption of residual alveolar bone and atrophy of jaw bones all accelerate the loosening and loss of teeth and severely affect the important functions of jaw bones, including maintaining the facial contour, supporting speech articulation, and mastication. Therefore, the prevention and treatment of age-related osteoporosis and tooth loss in the elderly population will face great challenges. Currently, the restoration and treatment approaches to age-related diseases, age-related osteoporosis and tooth loss all have shortcomings. Therefore, it is of great significance to study the regulation mechanism of differentiation and regeneration of mesenchymal stem cells under aging conditions for the repair and treatment of bone and tooth tissues in elderly patients and for the improvement of their quality of life.

Current treatment of osteoporosis is mainly aimed at preventing fracture, with bisphosphonates being the most commonly used drugs in clinical. Although bisphosphonates can reduce the incidence rate of fractures by 40-70%, they may cause side effects such as acute renal failure, oesophageal cancer, and musculoskeletal pain. In addition, long-term use of bisphosphonates increases the fracture risk in patients, especially typical femur fractures and osteonecrosis of jaw bones. Other drugs are anabolic agents, which are used to stimulate bone formation and reduce the fracture risk in patients. Parathyroid hormone (PTH) is the only drug approved by the US Food and Drug Administration to stimulate bone formation, but it relates to the development of osteosarcoma, and can only be used for 2 years. Postmenopausal women are considered to be at risk for accelerated bone loss [29], and hormone replacement therapy (oestrogen and progesterone therapy, oestrogen-only therapy, or selective oestrogen receptor modulators) remains the first-line option for clinical management. However, hormone therapy can increase the risk of breast cancer in patients. Calcium and vitamin D supplements are only suitable for preventing osteoporosis and are not completely effective in preventing the development of osteoporosis. Currently, although there are many drugs available for the treatment of osteoporosis, they have obvious side effects, limited bone recovery and variations in drug response among patients. There is still room for improvement in the clinical effect by giving drug treatment alone. Therefore, understanding the etiology and molecular mechanisms of osteoporosis may help to find more effective treatments that can prevent the deterioration of bone microstructure and maintain bone homeostasis.

Dental tissue defect and tooth loss are common and frequent diseases in the elderly population, seriously affecting chewing, speech, appearance and mental health. Existing restoration methods of tooth loss are non-biological prosthetic restorations, which are costly and often damage adjacent healthy teeth, and the teeth restored thereby are significantly different from natural teeth. Therefore, dental tissue regeneration has become a hot spot in international dentistry research. Mesenchymal stem cells have the ability to repair damaged tissue and have multi-differentiation potential, which can differentiate into all types of mesodermal cells. Therefore, stem cell-based tissue engineering technology has become an important means of repairing a wide range of tissue damage. However, like other body cells, the functions of stem cells in repairing damage, renewal and differentiation will diminish or even become dysfunctional with cell aging, which affects the therapeutic effect of stem cells. Restoring the function of senescent autologous mesenchymal stem cells in elderly patients may enhance the repair and regeneration potential of senescent tissues and avoid the immune rejection of allogeneic mesenchymal stem cells. Therefore, elucidating the mechanism of directed differentiation of senescent mesenchymal stem cells is the key to tissue repair and regeneration, and is of great significance for the repair and treatment of bone and dental tissues in the elderly.

### SUMMARY

In view of this, the present disclosure investigates the relationship between KDM6B, WDR5 and MLL1, as well as their roles and mechanisms in the regulation of senescence and osteogenic/odontogenic differentiation of mesenchymal stem cells.

In order to achieve the above object, the present disclosure provides the following technical solutions.

The present disclosure provides use of a protein complex as a target in the manufacture of an agent or a medicament for inhibiting senescence of mesenchymal stem cells, promoting osteogenic differentiation or odontogenic differentiation of mesenchymal stem cells;
wherein the protein complex is selected from the group consisting of a first protein complex and a second protein complex;
wherein, the first protein complex comprises WDR5 and KDM6B, and
the second protein complex comprises KDM6B, WDR5 and MLL1.

Most importantly, the present disclosure further provides a bioactive peptide, comprising:
(I) an amino acid sequence set forth in SEQ ID No: 1 or 2,
(II) an amino acid sequence derived from the amino acid sequence set forth in (I) by substitution, deletion or addition of one or more amino acids and having the same functions as the amino acid sequence set forth in (I), or
(III) an amino acid sequence having more than 90% identity to the amino acid sequence set forth in (I) or (II).

The present disclosure further provides a nucleic acid encoding the bioactive peptide.

Furthermore, the present disclosure further provides a biological material expressing the bioactive peptide, and the biological material comprises one or more of an expression vector, a plasmid, an expression cassette, a recombinant bacterium and a host cell.

Based on the above studies, the present disclosure further provides use of the bioactive peptide in the manufacture of an agent or a medicament for inhibiting senescence of mesenchymal stem cells, promoting osteogenic differentiation of bone marrow mesenchymal stem cells and/or promoting odontogenic differentiation of stem cells from apical papilla.

The present disclosure further provides use of the bioactive polypeptide in the manufacture of an agent or a medicament for preventing and treating osteoporosis and periodontitis, and/or for repairing damaged mucosa and skin.

In addition, the present disclosure further provides an agent or a medicament comprising the bioactive peptide and a pharmaceutically acceptable excipient.

From the perspective of clinical translational application, the present disclosure elucidates how to regulate the action of KDM6B/WDR5 under specific clinical conditions to reverse stem cell senescence and promote osteogenic/odontogenic differentiation of stem cells and bone/dental tissue repair and regeneration, based on the previous studies. The present disclosure also helps to elucidate the molecular regulatory mechanism of MSCs functioning in the microenvironment with aging and osteoporosis, which provides target genes and theoretical basis for the functional modification of MSCs and promotion of tissue regeneration. The present disclosure provides new small molecule agents as therapeutic drugs for aging and age-related diseases to promote bone/dental tissue regeneration and provides a basis for their clinical translational application. According to the study on the role and molecular mechanism of KDM6B, WDR5 and MLL1 signaling molecules in regulating the senescence and differentiation function of mesenchymal stem cells, it was found that:

WDR5 is a co-binding protein for negatively regulating the functions of histone demethylase KDM6B and MLL1. WDR5 can form a protein complex with histone demethylase KDM6B and inhibit the function of KDM6B to ultimately achieve the regulation of senescence and differentiation of stem cells and repair and regeneration of bone/dental tissue. Based on mechanism study, the present disclosure develops and utilizes small-molecule polypeptides targeting the binding region of KDM6B to WDR5 to regulate the function of mesenchymal stem cells by regulating the binding of KDM6B/WDR5 complex, ultimately restoring the function of mesenchymal stem cells under aging and osteoporotic conditions, thereby promoting the repair and regeneration of bone/dental tissues.

Included are, but not limited to:

### (I) Effect on Bone Marrow Mesenchymal Stem Cells:

1. WDR5 and KDM6B form a protein complex in bone marrow mesenchymal stem cells.
2. The formation of the protein complex of WDR5 and KDM6B is increased in aging bone marrow mesenchymal stem cells.
3. Bioactive polypeptides 102 and 114 can inhibit senescence of bone marrow mesenchymal stem cells and promote osteogenic differentiation of bone marrow mesenchymal stem cells.
4. Bioactive polypeptides 102 and 114 can inhibit senescence of bone marrow mesenchymal stem cells and promote osteogenic differentiation of bone marrow mesenchymal stem cells in aged mice.
   (II) Effect on Stem Cells from Apical Papilla
1. KDM6B, WDR5, and MLL1 form a protein complex in stem cells from apical papilla.
2. A mutation in binding site of the KDM6B/WDR5 protein enhances the function of KDM6B, and can thus inhibit the senescence of stem cells from apical papilla and promote their odontogenic differentiation.
3. Bioactive polypeptides 102 and 114 inhibit the senescence of stem cells from apical papilla and promote their odontogenic differentiation.

### (III) Use in Prevention and Treatment of Osteoporosis

Bioactive polypeptide 114 has the effect of enhancing bone mineral density in osteoporotic mice, and can effectively prevent bone loss in osteoporotic mice.

### (IV) Use in Treatment of Periodontitis

1. Bioactive polypeptide 114 can reduce periodontal pocket depth, and relief attachment loss and gingival recession in a minipig model of periodontitis.
2. Bioactive polypeptide 114 can promote the formation of new bone in alveolar bone in a minipig model of periodontitis.
3. Bioactive polypeptide 114 can promote repair of gingival soft tissue in a minipig model of periodontitis.

### BRIEF DESCRIPTION OF DRAWINGS

For more clearly illustrating embodiments of the present disclosure or the technical solutions in the prior art, drawings for describing the embodiments or the prior art will be briefly described hereinafter.

FIG. 1 shows the results of Co-IP (Co-immunoprecipitation) assay, indicating that WDR5 formed a protein complex with KDM6B and MLL1 in bone marrow mesenchymal stem cells; and after knockout of WDR5 in bone marrow mesenchymal stem cells, the protein complex of WDR5, KDM6B and MLL1 was reduced in the WDR5 knockout group, wherein β-actin was used as an internal reference.

FIG. 2 shows the results of Co-IP assay on the binding of WDR5 to KDM6B and MLL1 in bone marrow mesenchymal stem cells of 2-month-old and 18-month-old C57BL/6 mice, indicating that binding of WDR5 to KDM6B and MLL1 in bone marrow mesenchymal stem cells increased under aging condition: the protein complex of WDR5 and KDM6B, MLL1 increased in bone marrow mesenchymal stem cells of aged mice compared to young mice, wherein β-actin was used as an internal reference.

FIG. 3 shows the results of exploration of KDM6B-WDR5 protein binding sites using polypeptide microarray technology, wherein A shows that seven positive polypeptide binding sites are found after a polypeptide microarray chip based on KDM6B was produced and incubated with WDR5; B shows optical density of spots on the positive membrane of the array corresponding to KDM6B; C shows the results of Co-IP assay that indicates, among the three bioactive polypeptides of peptide 102, peptide 114 and peptide 152, only peptides 102 and 114 can effectively open the binding of KDM6B/WDR5 protein complex.

FIG. 4 shows that bioactive polypeptides 102 and 114 can inhibit senescence of bone marrow mesenchymal stem cells and promote osteogenic differentiation thereof. Wherein, A shows the result of ELISA assay of telomerase reverse transcriptase, indicating that peptide 114 can promote the expression of telomerase reverse transcriptase in bone marrow mesenchymal stem cells compared to the control group; B-C show the results of β-gal staining and quantification analysis, indicating that peptide 102 and peptide 114 lead to a significant reduction in the number of β-gal positive cells in bone marrow mesenchymal stem cells compared to the control group; D shows that peptide 102 and peptide 114 can increase the ALP activity of bone marrow mesenchymal stem cells compared to the control group; and E-F show the results of Alizarin red staining and quantification analysis of calcium ion, indicating that the mineralization capacity of bone marrow mesenchymal stem cells in the peptide 102 and peptide 114 groups is significantly higher compared to the control group. *P≤0.05, **P≤0.01.

FIG. 5 shows that bioactive polypeptides 102 and 114 can inhibit the senescence of bone marrow mesenchymal stem cells and promote their osteogenic differentiation. Wherein, A shows the results of β-gal staining and quantification analysis, indicating that peptide 102 and peptide 114 lead to a significant reduction in the number of β-gal positive cells in senescent bone marrow mesenchymal stem cells compared to the control group; B shows that peptide 102 and peptide 114 can increase the ALP activity of senescent bone marrow mesenchymal stem cells compared to the control group; and C-D show the results of Alizarin red staining and quantification analysis of calcium ion, indicating that the mineralization capacity of senescent bone marrow mesenchymal stem cells in the peptide 102 and peptide 114 groups is significantly higher compared to the control group. *P≤0.05, **P≤0.01.

FIG. 6 shows that the KDM6B forms a protein complex with WDR5 in stem cells from apical papilla; wherein, A shows the results of Co-IP assay, indicating that after knockout of KDM6B in stem cells from apical papilla, the protein complex of KDM6B and WDR5 is reduced in the KDM6B knockout group, with histone H3 as an internal reference; and B shows the results of Co-IP assay, indicating that after knockout of WDR5 in stem cells from apical papilla, the protein complex of WDR5 and KDM6B is reduced in the WDR5 knockout group, with β-actin as an internal reference.

FIG. 7 shows that the mutation in KDM6B sequence at sites 102 and 114 can inhibit senescence of stem cells from apical papilla and promote their odontogenic differentiation; wherein, A shows the results of Co-IP assay indicating that the binding of KDM6B to WDR5 is reduced in stem cells from apical papilla in HA-KDM6B-mut102 and HA-KDM6B-mut114 groups compared to the control group; B shows the results of ELISA assay of telomerase reverse transcriptase indicating that HA-KDM6B-mut114 can promote the expression of telomerase reverse transcriptase in stem cells from apical papilla compared to the control group; C-D show the results of β-gal staining and quantitative analysis indicating that the number of β-gal positive cells is reduced in stem cells from apical papilla in HA-KDM6B-mut102 and HA-KDM6B-mut114 groups compared to the control group; E shows that the ALP activity of stem cells from apical papilla in HA-KDM6B-mut102 group and HA-KDM6B-mut114 group is promoted compared to the control group; and F-G show the results of Alizarin red staining and quantitative analysis of calcium ion, indicating that the mineralization capacity of stem cells from apical papilla in the HA-KDM6B-mut102 and HA KDM6B-mut114 groups is significantly increased compared to the control group; *P≤0.05, **P≤0.01.

FIG. 8 shows that the bioactive polypeptides 102 and 114 can inhibit senescence of stem cells from apical papilla and promote their odontogenic differentiation; wherein, A shows the results of Co-IP assay indicating that the binding of KDM6B to WDR5 is reduced in stem cells from apical papilla in polypeptides 102 and 114 groups compared to the control group; B shows the results of ELISA assay of telomerase reverse transcriptase indicating that peptide 114 can promote the expression of telomerase reverse transcriptase in stem cells from apical papilla compared to the control group; C-D show the results of β-gal staining and quantitative analysis indicating that peptide 102 and peptide 114 lead to a significant reduction in the number of β-gal positive cells in stem cells from apical papilla compared to the control group; E shows that peptide 102 and peptide 114 increase the ALP activity of stem cells from apical papilla compared to the control group; and F-G show the results of Alizarin red staining and quantitative analysis of calcium ion indicating that the mineralization capacity of stem cells from apical papilla in peptide 102 and 114 groups is significantly increased compared to the control group; *P≤0.05, **P≤0.01.

FIG. 9 shows the result of measurement of new bone volume according to three-dimensional reconstruction of CBCT image at week 12 after injection of the bioactive polypeptide, indicating that new bone formation volume in the defect area with periodontitis in the 114 peptide group is higher than that in the control peptide group and the PBS group; and the difference in new bone volume between the groups is significant; *P≤0.05.

FIG. 10 shows the probing depth in the 114 peptide group was significantly lower than that in the PBS group and the control peptide group as measured by a periodontal probe at week 12 after injection of the bioactive polypeptide; and the difference between the 114 peptide and the rest of the groups is of significant significance; *P≤0.05.

FIG. 11 shows the attachment loss in the 114 peptide group was significantly lower than that in the PBS group and the control peptide group as measured by a periodontal probe at week 12 after injection of the bioactive polypeptide; and the difference between the 114 peptide and the rest of the groups is of significant significance; *P≤0.05.

FIG. 12 shows the gingival recession degree in the 114 peptide group was significantly lower than that in the PBS group and the control peptide group as measured by a periodontal probe at week 12 after injection of the bioactive polypeptide; and the difference between the 114 peptide and the rest of the groups is of significant significance; *P≤0.05.

FIG. 13 shows the results of three-dimensional reconstruction of DICOM images by using Mimics 17.0 image processing software after obtaining CBCT tomographic images by continuously scanning the natural occlusal position of the tested minipigs lying flat on the imaging table of the CBCT scanner with the head fixed. The CBCT data was obtained before and after modeling and after surgery for post-treatment evaluation. The postoperative three-dimensional reconstruction model shows that there was new bone formation in the PBS group, control peptide group and 114 peptide group; and the effect of new bone formation in the 114 peptide group was better than that in the control peptide group and the PBS group.

FIG. 14 shows that at week 12 after injection of the bioactive polypeptide, wounds in the bone defect area of periodontal tissues of minipigs in the PBS group, control peptide group and 114 peptide group exhibited healing, no infected and necrotic tissues were seen; and the restoration effect of soft tissues in the 114 peptide group was better than that in the control peptide group and the PBS blank control group.

FIG. 15 shows that bioactive polypeptide 114 promotes the healing of mouse defected palatal mucosa. After injected with PBS, the mice were injected with control peptide and 114 peptide at the same amount around the defected palatal mucosa, and changes in the healing of mucosa was observed. Wherein, a, b and c show the mice palatal defect model; d, e, and f show the defected palatal mucosa in PBS group, control peptide group and 114 peptide group on day 14 respectively; and g, h, and i show the defected palatal mucosa in PBS group, control peptide group and 114 peptide group on day 21 respectively. The results indicate that the defected palatal mucosa in 114 peptide group was obviously healed compared to PBS group and control peptide group.

FIG. 16 shows the area of unhealed defected palatal mucosa of mice in the PBS, control peptide, and 114 peptide groups on day 14 and day 21, and there was statistical significance in the 114 peptide group compared to the PBS and control peptide groups; *P≤0.05.

FIG. 17 shows that 114 peptide promotes the healing of defected dorsal skin of mice. The mice were injected with equal amounts of PBS, control peptide, and 114 peptide on the back around a full-thickness skin defect with a diameter of 6 mm, wherein, a and b show the defected skin of the mice in the PBS group on days 0 and 14, respectively; c and d show the defected skin of mice in the control peptide group on days 0 and 14; e and f show the defected skin of mice in the 114 peptide group on days 0 and 14, respectively. The results indicate a significant healing of the defected skin in the 114 peptide group compared to the PBS and control peptide groups.

FIG. 18 shows the healing rate of defected dorsal skin in mice in the PBS, control peptide and 114 peptide groups obtained by calculating on days 10 and 14, which indicates a statistical significance in the 114 peptide group compared to the PBS and control peptide groups; *P≤0.05, **P≤0.01, ***P≤0.001.

FIG. 19 shows that bioactive polypeptide 114 can prevent femoral bone loss in osteoporotic mice; wherein A shows the change in the bone trabeculae of mice as observed by subjecting metaphysis of the mouse femur to Micro-CT; B shows the Micro-CT analysis of metaphysis of the mouse femur, indicating that there was no significant difference in bone density of femoral bone trabecular between the mice injected with the PBS and control peptide and the mice injected with OVX; the bone density of femoral bone trabeculae in OVX group, PBS group and control peptide group showed a significant reduction compared to sham group; and the mice in 114 peptide group showed a significant up-regulation in trabecular bone density compared to OVX group, PBS group and control peptide group.

### DETAILED DESCRIPTION

The present disclosure discloses a polypeptide sequence of KDM6B and use thereof in the regulation of the function of mesenchymal stem cells. Those skilled in the art can learn from the contents of the present disclosure and appropriately improve the processing parameters. It should be particularly indicated that, all similar replacements and changes are obvious for those skilled in the art, which are deemed to be included in the present disclosure. The method and the application of the present disclosure have been described through the preferred embodiments, and it is obvious that the method and application described herein may be changed or appropriately modified and combined to realize and apply the technology of the present disclosure by those skilled in the art without departing from the content, spirit and scope of the present disclosure.

KDM6B, acting as a demethylase of H3K27me2/3, can play an important role in the development of bone and tooth tissues by affecting mesenchymal stem cell-specific lineage differentiation. KDM6B is also involved in bone resorption. There is a study showing that increased differentiation into osteoclasts after bone injury can lead to osteoporosis, in which KDM6B reduces the methylation level of H3K27me3 in the Nfatc1 gene, and activates expression of Nfatc1 gene to achieve the maintenance of bone mass, thus preventing osteoporosis. In addition, the down-regulation of expression of the histone demethylase KDM6B can lead to apoptosis and an increase in senescent cells, and this senescence results in the loss of self-renewal capacity of adult stem cells. In view of this, it is conjectured that KDM6B plays an important role in the senescence and dysfunction of mesenchymal stem cells. In the present disclosure, the effect of KDM6B on senescence of mesenchymal stem cells is investigated. The experimental results show that KDM6B can inhibit the expression of β-gal and p16^{INK4A} and up-regulate the activity of telomerase reverse transcriptase in bone marrow mesenchymal stem cells and stem cells from apical papilla. Senescence-associated-β-galactosidase (SA-β-gal) is one of the earliest biomarkers for the identification of senescent cells in cultured cells and fresh tissue samples, and this marker effectively demonstrates that senescent cells progressively accumulate in senescence-associated disease foci and senescent tissues in a variety of mammals. Another distinctive feature of senescent cells is the increased expression of cell cycle inhibitory proteins, which leads to the maintenance of a stagnant state of the senescent cells and consequently leads to the accumulation of senescent cells, wherein p16^{INK4A} is the predominant cell cycle inhibitory protein. Quercetin and fisetin have now been proven to stimulate tissues and cells against ageing under a variety of conditions in vitro and in vivo. Most notably, both of quercetin and fisetin can effectively reduce the expression of p16 and SA-β-gal. In addition, it is believed that the inhibition of telomere shortening as a measure to prevent and reduce cellular senescence is a reliable therapeutic approach in the context of aging and telomere dysfunction. Systemic delivery of telomerase reverse transcriptase (Tert) can reduce a number of senescence markers, improve conditions associated with aging and extend the lifespan of wild-type mice, thus demonstrating that the maintenance of telomere function plays a role in natural aging.

According to the literature and the preliminary experimental data, it is suggested that KDM6B has the function of promoting the directed osteogenic/dentogenic differentiation of mesenchymal stem cells and inhibiting their senescence. Therefore, KDM6B is a candidate target for the prevention and treatment of age-related diseases due to its role in ageing and bone/dental tissue regeneration. However, the effective regulation of KDM6B function is not clear, and its regulatory mechanism needs to be thoroughly investigated. It is found that WDR5 can form a protein complex with KDM6B in HEK293 cells according to literatures. In order to verify this result, both bone marrow mesenchymal stem cells and stem cells from apical papilla were subjected to Co-IP assay. The results show that WDR5 can form a protein complex with KDM6B in both cells. In aging animal models, C57 mice aged between 18 to 24 months highly correlate with human aged from 56 to 69, and may more accurately simulate the state of senescent mesenchymal stem cells. Therefore, bone marrow mesenchymal stem cells from 2 and 18 months old mice were subjected to Co-IP assay. The results show an increased amount of protein complex of KDM6B and WDR5 in bone marrow mesenchymal stem cells from aged mice compared to young mice. WDR5 is involved in the regulation of various cellular physiological activities, such as epithelial mesenchymal transition, leukaemogenesis, differentiation of chondrocytes and osteoblasts, and maintenance of multipotency of embryonic stem cells. Moreover, WDR5 and the methylation of histones play a very important role in the growth and development of vertebrates. However, there is a lack of certain studies on the effects of WDR5 on osteogenic/odontogenic differentiation of mesenchymal stem cells and on senescence. Based on the above results, it is speculated that WDR5 may negatively regulate the action of KDM6B on mesenchymal stem cells, and the special protein structure of WDR5 may play a central scaffolding role in forming a complex with multiple proteins to regulate stem cell differentiation, proliferation and antiviral effects. It has been found that WDR5 is a key co-binding protein that catalyses the activity of H3K4me3 transferase and MLL1 complex. Therefore, when investigating the role of WDR5 in regulating stem cells, the effect of MLL1 thereon is also in great need of attention. Based on the above results, it is suggested that WDR5 may be a negative regulator of the function of KDM6B and MLL1 on regulating mesenchymal stem cells. To confirm this conjecture, seven binding sites for KDM6B/WDR5 protein complex were found using protein microarray technology, including:

| | |
|---|---|
| 37 | RESRVQRSRMDSSVS (as set forth in SEQ ID No. |
| | 3); |
| 93 | CETLVERVGRSATDP (as set forth in SEQ ID No. |
| | 4); |
| 102 | FITC-(Acp)-KEKSRRVLGNLDLQSYGRKKRRQRRR (as |
| | set forth in SEQ ID No. 1); |
| 114 | FITC-(Acp)-ADLTISHCAADVVRAYGRKKRRQRRR (as |
| | set forth in SEQ ID No. 2); |
| 128 | SRSHTTIAKYAQYQA (as set forth in SEQ ID No. |
| | 5); |
| 152 | FITC-(Acp)-IVPMIHVSWNVARTVYGRKKRRQRRR (as |
| | set forth in SEQ ID No. 6); and |
| 153 | VARTVKISDPDLFKM (as set forth in SEQ ID No. |
| | 7). |

Wherein, three sites of 102, 114, and 152 showed the highest degree of binding. Subsequently, biologically active polypeptides were constructed and subjected to screen of the optimal concentration for cell stimulation. 10 μg/mL was screened out by in vitro ALP assay to be the optimal concentration. Co-IP assay using peptides 102, 114 and 152 to stimulate cells revealed that 152 could not effectively block the binding of KDM6B and WDR5. In order to further exclude the effect of both FITC fluorescent sequence and cell-penetrating peptide attached to the tested polypeptides on the cells, a control polypeptide group was set. The Co-IP results show that peptides 102 and 114 can block the formation of the KDM6B/WDR5 complex. On this basis, it is suggested that blocking the formation of the KDM6B/WDR5 protein complex may enhance the functional role of KDM6B, thereby restoring the ability of stem cells to repair and regenerate tissue under senescent conditions. Interestingly, the above conjecture was confirmed by the results of experiments on senescence and osteogenic/odontogenic differentiation in vitro. The polypeptides 102, and 114 for blocking KDM6B/WDR5 can enhance the ability of bone marrow mesenchymal stem cells and stem cells from apical papilla to achieve directed osteogenic/dentogenic differentiation and anti-aging. Moreover, polypeptides 102 and 114 for blocking KDM6B/WDR5 can rescue the osteogenic differentiation capacity of senescent mouse bone marrow mesenchymal stem cells and reduce the expression of senescence markers of β-gal and P16. To further confirm that the 102 and 114 sequences can block the formation of KDM6B/WDR5 protein complex, HA-tagged KDM6B plasmid with the sequences of 102 and 114 removed was transfected into stem cells from apical papilla, and the Co-IP assay was carried out, which further confirmed that the mutation of the 102 and 114 peptide sequences can block the formation of KDM6B/WDR5 complex. Both fragments enhance the ability of KDM6B to promote osteogenic/odontogenic differentiation and anti-aging of mesenchymal stem cells.

In conclusion, it is suggested that WDR5 may be a co-binding protein that negatively regulates the functions of histone demethylases KDM6B and MLL1 in the study. On the basis of the mechanism study, according to a binding region sequence of KDM6B to WDR5, small-molecule polypeptides were developed, and utilized to regulate the function of the mesenchymal stem cells by regulating and controlling the binding of a KDM6B/WDR5 complex. Ultimately, the function of mesenchymal stem cells was restored under aging conditions, thus the repair and regeneration of bone/dental tissues can be promoted. Further studies reveal that the small molecule polypeptides have a preventive effect against osteoporosis and periodontitis.

### Experimental Cells

Stem cells from apical papilla (SCAPs) were obtained from orthodontic teeth or third molars with impaction extracted in the outpatient clinic of Oral Maxillofacial and Alveolar Surgery of Beijing Stomatological Hospital affiliated to Capital Medical University, which all were collected with the informed consent of the patients (16-22 years old) who met the requirement of being free of systemic diseases. The collected teeth were free of dental and periodontal diseases, and were subjected to primary cell culture.

Human bone marrow mesenchymal stem cells (BMSCs) were purchased from ScienCell.

C57BL/6 mouse bone marrow mesenchymal stem cells were obtained from the culture of primary cells from 2-month-old and 18-month-old C57BL/6 mice.

293T cells were purchased from Suzhou Gemma Genetics Co. SCAPs, and BMSCs used for experiments were 3rd-5th generation cells.

### Experimental Animals

Male C57BL/6 mice aged 2 months and 18 months were purchased from Speifu (Beijing) Biotechnology Co., Ltd.

### Main Equipments

| Equipment | Brand/Suppler | Origin |
|---|---|---|
| Ultra-clean bench | Harbin Donglian | China |
| | Electronic Technology | |
| | Development Co., Ltd. | |
| Motorized pipette | EPPENDORF | Germany |
| Centrifuge | Sigma | Germany |
| Sterile centrifuge tubes | Corning | United States |
| | | of America |
| Petri dish | Corning | United States |
| | | of America |
| Sterile pipette | Corning | United States |
| | | of America |
| 6-well plate | Corning | United States |
| | | of America |
| 96-well plate | Corning | United States |
| | | of America |
| CO₂ incubator | Heal Force 90 Shanghai | China |
| PCR quantitative | BIO-RAD | United States |
| instrument | | of America |
| PCR amplifier | BIO-RAD | United States |
| | | of America |
| Electronic balance | Sartorius | Germany |
| Spectrophotometer | Thermo Scientific | United States |
| | | of America |
| −20° C./4° C. | SIEMENS | Japan |
| refrigerator | | |
| −80° C. low temperature | Thermo Scientific | United States |
| refrigerator | | of America |
| Inverted microscope and | OLYMPUS TH4-200 | Japan |
| imaging system | | |
| Benchtop high-speed | Thermo Scientific MR23 | United States |
| refrigerated centrifuge | | of America |
| Microplate reader | MolecuLes Devices | United States |
| | | of America |
| Electrothermal | Beijing Changfeng Industry Co., | China |
| thermostatic water bath | Ltd. | |
| Ultrasonic cell pulveriser | Shanghai Zhixin Instrument Co., | China |
| | Ltd. | |
| Cell counter | BIO-RAD | United States |
| | | of America |

### Main Reagents

| Reagent | Brand/Suppler | Origin |
|---|---|---|
| a-MEM medium | Invitrogen | United States |
| | | of America |
| MSCM medium | ScienCell | United States |
| Osteogenic induction medium | Invitrogen | of America |
| | | United States |
| | | of America |
| Fetal bovine serum (FBS) | GIBCO | Germany |
| PBS | GIBCO | United States |
| | | of America |
| Cell freezing solution | NCM Biotech | China |
| IP lysis buffer | SIGMA | United States |
| | | of America |
| Anti-HA monoclonal | Cell Signaling Technology | United States |
| antibody | | of America |
| Anti-Myc monoclonal | Cell Signaling Technology | United States |
| antibody | | of America |
| WDR5 monoclonal antibody | Santa Cruz Biotechnology | United States |
| | | of America |
| KDM6B antibody | Millipore | United States |
| | | of America |
| MLL1 polyclonal antibody | ABclonal | China |
| Anti-normal IgG | Santa Cruz Biotechnology | United States |
| | | of America |
| Histone H3 monoclonal | Santa Cruz Biotechnology | United States |
| antibody | | of America |
| P16 | Cell Signaling Technology | United States |
| | | of America |
| β-actin monoclonal | ABclonal | China |
| antibody | | |
| DAPI | Solarbio | China |
| ChIP kit | Merck Millipore | United States |
| | | of America |
| PIC | SIGMA | United States |
| | | of America |
| PMSF | SIGMA | United States |
| | | of America |
| Lysis buffer | SIGMA | United States |
| | | of America |
| WB luminescent solution | BIO-RAD | United States |
| | | of America |
| RNA extraction kit | QIANGEN | China |
| Polybrene | SIGMA | United States |
| | | of America |
| Trizol | Invitrogen | United States |
| | | of America |
| 0.25% Trypsin | Invitrogen | United States |
| | | of America |
| Double antibiotics | Invitrogen | United States |
| (Streptomycin | | of America |
| and Penicillin) | | |

### Statistical Analysis

Statistical analysis was performed using SPSS 19.0 statistical software. T-test was used to compare the measured data of two groups, and ANOVA analysis was used to compare the measured data of multiple groups, based on a statistical difference of P≤0.05.

TABLE 1
Abbreviation/symbol

| Abbreviation | Full name |
|---|---|
| ALP | Alkaline phosphatase |
| BMSCs | Bone mesenchymal stem cells |
| BSP | Bone sialoprotein |
| Co-IP | Co-Immunoprecipitation |
| DMSO | Dimethyl sulfoxide |
| DSPP | Dentin sialophosphoprotein |
| FBS | fetal bovine serum |
| KDM6B | Lysine(K)-specific demethylase 6B |
| KDM3B | Lysine (K)-Specific demethylase 3B |
| MSCs | Mesenchymal stem cells |
| MLL1 | Mixed Lineage Leukemia 1 |
| OCN | Osteocalcin |
| OSX | Osterix |
| OPN | Osteopontin |
| PBS | Phosphate buffered saline |
| RUNX2 | Runt-related transcription factor 2 |
| SCAPs | Apical papilla stem cells |
| TERT | Telomerase Reverse Transcriptase |
| WDR5 | WD repeat domain 5 |
| β-gal | β-Galactosidase |
| α-MEM | α-minimal essential medium |

The present disclosure provides a polypeptide sequence of KDM6B and use thereof in the regulation of mesenchymal stem cell function, wherein the used raw materials and reagents are commercially available.

The present disclosure is further illustrated below in combination with examples.

### EXAMPLES

### Example 1 Acquisition, Isolation and Culture of Mesenchymal Stem Cells

(1) Isolation and Culture of Human Stem Cells from Apical Papilla

After getting informed consent of patients, orthodontic teeth orthodontic teeth or third molars were aseptically extracted from patients under local anaesthesia, and the extracted tooth was placed into PBS containing double antibiotics in a pre-prepared sterile centrifuge tube. In an ultra-clean bench, apical papilla tissue from the root end of tooth was collected by scraping using a sterile blade. The collected apical papilla tissue was repeatedly washed with a large volume of PBS containing double antibiotics, then placed in a digestive solution containing collagenase type I (3 g/L) and dispase (4 g/L) in a ratio of 1:1, cut into pieces, and digested for 40 minutes in an incubator at 37° C. Subsequently, 2 times the volume of culture medium was added to terminate the digestion. The cells were collected into 15 mL sterile centrifuge tubes, and centrifuged at 1100 rpm for 6 minutes. The supernatant was removed, and the cells pellet was resuspended in culture medium, mixed well by pipetting up and down, seeded in 60 mm petri dishes, and incubated in an incubator at 37° C. with 5% CO₂. After 3 days of culture, the growth status of cells was observed under a microscope, and the cells were replaced with fresh culture medium. When the cells grew to about 80-90% confluence, they were digested using 0.25% trypsin and passaged in 100 mm petri dishes at a ratio of 1:2.

(2) Isolation and Culture of Bone Marrow Mesenchymal Stem Cells from C57BL/6 Mice.

After sacrificed by cervical dislocation, the mice were soaked in 75% alcohol for 5 minutes. Then the skin of the limbs was peeled off in the ultra-clean bench, the muscles and fascia on the surface of tibia, femur and humerus were carefully removed, and the joints at both ends of the long bones were clipped using ophthalmic scissors. The bone marrow in the long bones was flushed into a 5 cm petri dish with α-MEM medium at a constant temperature of 37° C. extracted with a 1 ml syringe with a No. 2 needle. The bone marrow was incubated at 37° C. under 5% CO₂ for 5 days, and then cell clones were observed. Then the cells were passaged into a large dish using 0.25% trypsin, and the culture solution was changed every 2-3 days. Cell growth was observed under an inverted microscope every day. When the cells grew to 80% confluence, the cells were digested using trypsin according to a ratio of 1:3 and passaged.

### Example 2 Cell Culture and Induction of Osteogenic/Odontogenic Differentiation

Mesenchymal stem cells were cultured in ScienCell mesenchymal stem cell culture medium in a 37° C., 5% CO₂ cell incubator. Primary cells were cultured and passaged to 3-5 generations for cell experiments. When the cells were ready, the cells were digested with trypsin into single cells, and 3.0×10⁵ cells were counted by a cell counter and plated in a 6-well plate. Induction of osteogenic differentiation was performed after the number of cells grew to 80-90%. The culture solution was changed once every three days.

### Example 3 Cryopreservation and Thaw of Mesenchymal Stem Cells

### (1) Cryopreservation

The cell culture medium was replaced one day before cryopreservation of cells. The laboratory supplies used, such as gun tips, pipettes and centrifuge tubes, were sterilized in an ultra-clean bench for 30 minutes in advance. After washed twice with PBS, the cells were digested with 0.25% trypsin at 37° C. for 2 minutes. After the cells were observed to float and become single cells under an inverted microscope, the digestion was terminated by adding 3 times the volume of culture medium. The digested cells were pipetted and mixed well to a suspension, transferred to a 15 mL centrifuge tube and centrifuged at 1100 rpm/min for 6 minutes. The supernatant in the centrifuge tube was removed, and the cryopreservation solution was added. After mixed well and dispensed into freezing tubes, the cells were placed in a −80° C. refrigerator overnight and then putted in a liquid nitrogen tank for long-term storage. Cell name, generation and date were labeled on freezing tubes.

### (2) Thaw

The laboratory supplies used, such as gun tips, pipettes and centrifuge tubes, were sterilized in an ultra-clean bench for 30 minutes in advance. The culture medium was taken out of a refrigerator and placed in an incubator to be preheated. A mask and gloves were worn, and the cells stored in liquid nitrogen were taken out and quickly placed in a 37° C. water bath under constant shaking to be thawed. The thawed cells were taken out of the water bath. The freezing tube was opened in an ultra-clean bench disinfected by 75% alcohol, and the cell suspension was pipetted and transferred into a centrifuge tube containing culture medium, and centrifuged at 1100 rpm for 6 minutes. The supernatant was discarded, and the cells were added with culture medium, mixed well by pipetting, seeded into petri dishes, and cultured in an incubator with 5% CO₂ at 37° C.

### Example 4 Viral Packaging and Cell Transfection

### (1) CONSTRUCTION OF VIRAL PLASMIDS

The gene sequences of KDM6B, WDR5 and MLL1 were searched on https://www.ncbi.nlm.nih.gov/, the NCBI database platform. The siRNAs of KDM6B, WDR5 and MLL1 were designed using the program provided by Whitehead, which were inserted into a lentiviral shRNA vector pLKO.1, and sequenced for identification, and finally plasmids of KDM6B shRNA, WDR51shRNA and MLL1shRNA were successfully constructed. The full length of KDM6B gene with surface HA tag and WDR5 gene with Myc tag was obtained by gene synthesis, and both genes were connected to an expression vector of retrovirus PQCXIN, and sequenced for identification, and finally overexpression plasmids of KDM6B and WDR5 were successfully constructed. The full-length KDM6B gene with surface HA tag was obtained by gene synthesis, and the sequences at 102 and 114 sites were removed. Then the obtained sequence was connected to the expression vector of retrovirus PQCXIN, and sequenced for identification, and finally an overexpression plasmid of KDM6B with mutation at 102 and 114 sites was successfully constructed. PQCXIN was used as an empty vector control.

### (2) Viral Packaging

293t cells were transfected with control Scramble shRNA (Scramsh), KDM6B shRNA (KDM6Bsh), WDR5 shRNA (WDR5sh), MLL1 shRNA (MLL1sh) and the corresponding packaging plasmids (VSVG and DV-8.2). 48 hours after the transfection, the supernatant was collected, and the virus titer was identified. The cells were aliquoted and stored in a refrigerator at −80° C. 293T cells were transfected with control retrovirus empty plasmids PQCXIN, PQCXIN-HA-KDM6B, PQCXIN-HA-KDM6B-mut102, pqcxin-ha-kdm6b-mut14, PQCXIN-Myc-WDR5 and corresponding packaging plasmids (VSVG and GPZ). 72 hours after the transfection, the supernatant was collected, and the virus titer was identified. The cells were aliquoted and stored in a refrigerator at −80° C.

### (3) Establishment of Stable Transgenic Cells

Cells were seeded in Petri dishes. When the cells grew to a density of 50%-60%, the cells were replaced with 6 mL of culture medium, and added with 6 μg/mL Polybrene. Then the cells were transfected with Scramsh, KDM6Bsh, WDR5sh and MLL1sh viruses respectively. 12 hours after the transfection, the cells were replaced with fresh culture medium. 48 hours after the transfection, the cells were screened with puromycin for 3 days to obtain stable transgenic cells of control Scramsh, knockout of KDM6B, WDR5, and MLL1. The knockout effect was detected at protein and RNA levels. Control plasmid PQCXIN, PQCXIN-HA-KDM6B, PQCXIN-HA-KDM6B-mut102, PQCXIN-HA-KDM6B-mut114, and PQCXIN-Myc-WDR5 viruses were transfected into cells. 48 hours after the transfection, the cells were screened with G418 for 7 days to obtain the stable transgenic cells. The expression of exogenous KDM6B and WDR5 were detected at protein and RNA levels to obtain the stable transgenic cells overexpressing KDM6B, KDM6B-mut102, KDM6B-mut114 and WDR5.

### Example 5 Detection on the Changes in Protein Expression by Western Blot

### (1) Extraction of Total Protein

Before the end of cell culture, the experimental reagents, experimental materials and ice were prepared in advance. The culture medium in the petri dish was discarded, and the cells were washed 3 times with 5 mL of PBS pre-cooled at 4° C. A lysate was prepared with RIPA, PMSF and PIC in a ratio of 100:1:1, and added to the petri dish at an amount determined according to the specific situation (usually 500 μL for a 10-cm petri dish). Then the cells were incubated in a 4° C. refrigerator for 20 minutes, and the lysate in the petri dish was shaken every 5 minutes to cover the bottom of the petri dish. The cells were scraped and transferred into a 1.5 mL centrifuge tube, and centrifuged at 4° C. and 14000 rpm for 15 minutes. The supernatant was transferred into a 1.5 mL EP tube, labeled and stored at −80° C.

### (2) Measurement of Protein Concentration by Bradford Method

The protein sample stored at −80° C. was taken, and thawed quickly on ice. 200 μL of 1× Kaumas Brilliant Blue (Biobad) was added into each well of a 96-well plate, and 1 μL of protein sample was added into each well (the loading amount of protein was determined according to the color change), and mixed evenly. After bubbles were eliminated, the plate was detected for OD value on a machine. A standard curve was plotted accordingly. The sample was loaded at an equal volume and equal mass. The sample was loaded at 25 μg (the required volume of protein was calculated). The protein sample was diluted with PBS+PMSF+PIC to 20 μL, and added with 5 μL of 5× loading buffer. The protein was denatured at 95-100° C. for 10 minutes, then placed on ice for 10 minutes, and then stored at −20° C.

### (3) Electrophoresis

A precast gel was taken. The bottom tape was removed. The gel and the backing plate were correctly assembled (both font fronts facing the experimenter). A running buffer was added, and the recoverable running buffer was added to the outer chamber according to the scale until full, and then the comb was removed. The comb of precast gel was gently removed. The samples were added to each well, and 8 μL of Marker was added next to the protein sample wells. The electrophoresis was performed on the concentrated gel at 80V for 40 min and on the gradient gel at 120V until Marker reached the black line at the bottom, and then the power was turned off.

(4) Transfer Membrane without Touching Water

The precast gel was taken out, and the concentrated gel and part of the bottom gel were removed. The remaining bottom gel was covered with a PVDF membrane on its surface (labeling at a position). A filter paper, PVDF membrane, gel, and another filter paper were assembled in order onto a transfer module. Air bubbles were removed, and a cassette lid was covered and closed tightly. Electrotransfer was performed at constant voltage of 1.3 V for 7 minutes, and the power was turned off. The PVDF membrane was quickly taken out, and washed three times with 1×TBST, each time 5 min. Then the PVDF membrane was blocked: 5% skimmed milk in 1×TBST was prepared as the blocking solution. The PVDF membrane was placed in the blocking solution, incubated at room temperature for 1 hour, and washed four times with 1×TBST on a shaker, 10 minutes each time.

(5) Incubation with Primary and Secondary Antibodies

According to the molecular weight difference between the internal reference and the target protein, the way to incubate with antibody was determined. When the difference is more than 5 Kda, the antibody was incubated after cutting the PVDF membrane; and when the difference is less than 5 Kda, the antibody was incubated in two times. The membrane was placed in TBST milk containing the appropriate concentration of primary antibody (including GAPDH, HSP90 and other internal reference that may be needed after membrane cutting), and shaken at 4° C. overnight. After incubation with the primary antibody, the membrane was washed 3 times with 1×TBST with 5 minutes each time. The secondary antibody was diluted at 1:2000 and the incubation was performed on the shaker at room temperature for 1 hour. The PVDF membrane was washed three times with 1×TBST with 5 minutes each time.

### (6) Developing

The luminescent solution was prepared by mixing at 1:1 in a darkroom in advance. The PVDF membrane was placed in the dark box and the cut membrane were reinstated. The luminescent solution was added dropwise on the PVDF membrane (the area containing protein). The red light was used for excitation for 2-3 minutes, and imaging was performed using the BIO-RAD imaging system.

### Example 6 Co-Immunoprecipitation (Co-IP) of Protein

(1) Extraction of total proteins from cells

Protein was extracted from cells of knockout group and overexpression group using IP lysate. The knockout group cells SCAPs-Scramsh and SCAPs-KDM6Bsh, SCAPs-Scramsh and SCAPs-WDR5sh, BMSCs-Scramsh and BMSCs-WDR5sh were added with KDM6B antibody, WDR5 antibody, and IgG antibody, respectively, and then rotated on a shaker at 4° C. overnight. On the next day, the cells were added with Protein A/G beads and rotated on a rotary shaker at 4° C. for 2 hours. A complex of protein-antibody-agarose bead was formed, and the formation of a protein complex of KDM6B and WDR5WB was detected by WB. The overexpression group cells SCAPs-Vector, SCAPs-HA-KDM6B, SCAPs-HA-KDM6B-mut102, and SCAPs-HA-KDM6B-mut114 were added with ProteinA/G beads with HA antibody, and cells SCAPs Vector and SCAPs-Myc-WDR5 were added with ProteinA/G beads with Myc antibody. All cells were incubated overnight to form a complex of protein-antibody-agarose bead. The formation of a protein complex of KDM6B and WDR5 was detected by WB method.
(2) Grouping: Input group (loading amount of 25 μg); antibody group (loading amount of 800 μg, 2 μg of antibody); IgG group (loading amount of 800 μg, 2 μg of antibody). Samples were diluted with lysate.
(3) Preparation of protein A/G beads

The protein A/G beads were washed 4 times with PBS, and centrifuged at 2000 g for 2 minutes. The supernatant was carefully collected, and added with PBS to achieve a content of protein A/G beads of 50%. 30 μL of protein A/G beads was added to each tube, and rotated at 4 degrees overnight.
(4) The samples were centrifuged at 5000 rpm for 30 s at 4° C., and washed 4-5 times with PBS by inverted up and down. Then the samples were centrifuged, and the supernatant was discarded. The samples were added with 30 μL of 2× loading buffer, and placed at 100° C. for 5 minutes.
(5) The supernatant was subjected to electrophoresis.

### Example 7 Osteogenic/Odontogenic Induction

(1) Osteogenic culture medium was purchased from Invitrogen, USA.
(2) Quantification of alkaline phosphatase (ALP) activity
1) After 3 days of osteogenic induction, the culture medium was discarded, and the cells were washed with PBS twice.
2) The cells were added with 500 μL of lysis buffer, and incubated at 37° C. for 15 minutes.
3) The cells were scraped, transferred into a 1.5 mL EP tube, and centrifuged at 14000 rpm at 4° C. for 10 minutes. The supernatant was transferred into a new EP tube to measure the protein concentration, and the procedure was the same as that in Western blot.
4) The capsule from the alkaline phosphatase kit (Stock substrate Sol.) was dissolved with 5 mL of distilled water under vigorous shaking, and mixed well.
5) 50 μL of ALP buffer was added to 50 μL of Stock substrate Sol to obtain a mixture. 100 μL of the mixture was added to each well in a 96-well plate, then added with 10 μL of sample, and mixed well. A blank well without sample added was set for zero adjustment.
6) The plate was incubated at 37° C. for 15 minutes, and the OD value was measured at 405 nm.

Y=18.904*X−0.2817 (X=OD value)

Result of ALP activity was calculated according to Y/incubation time/protein concentration.
(3) Alizarin red staining
1) After two weeks of osteogenic induction, the culture medium was discarded. The cells were washed three times with PBS pre-cooled at 4° C., and fixed with 70% ethanol at 4° C. for 1 hour.
2) The cells were washed 2 times with double distilled water, and stained with 40 mM alizarin red solution (pH 4.2) at room temperature for 10 minutes. The staining was observed by naked eyes.
3) The cells were washed 3 times with double-distilled water.
(4) Determination of Ca²⁺ concentration

To quantitatively determine the concentration of calcium, the cells were added with 10% cetyl pyridinium chloride for dissolution for 30 minutes at room temperature. The OD value was measured at 562 nm using a spectrophotometer, and the calcium ion concentration of the samples was calculated according to the standard curve of calcium ion concentration, with the total protein concentration as an internal reference.

### Example 8 Cell Senescence-Specific β-Galactosidase Staining

Prior to the start of the experiment, the GENMED staining solution (Reagent E) in the kit was taken out of the refrigerator at −20° C., and thawed in an ice bath. Then 9.5 mL of GENMED dilution (Reagent D) was added into a 15 ml conical centrifuge tube, added with 500 μL of GENMED staining solution (Reagent E), mixed well, and then preheated in a 37° C. thermostatic sink. The obtained mixture was labeled as GENMED staining working solution. Then the following operations were performed:
(1) The culture solution was carefully removed from the 24-well cell culture plate.
(2) 500 μL of GENMED clean-up solution (Reagent A) was added to each well to clean the surface of growing cells.
(3) The clean-up solution in each well was carefully removed.
(4) 500 μL of GENMED fixing solution (Reagent B) was added to each well to cover the entire growing cell surface.
(5) The cells were incubated for 5 minutes at room temperature.
(6) The fixing solution was carefully removed.
(7) 500 μL of GENMED acidic solution (Reagent C) was added to each well to wash the cell surface;
(8) The acidic solution was carefully removed.
(9) Steps 7 and 8 were repeated once.
(10) 400 μL of preheated GENMED staining working solution was added to each well to cover the entire cell surface.
(11) The cells were incubated in a 37° C. incubator for 3-16 hours, or until the cells showed a blue color (Note: Evaporation of the liquid should be avoided).
(12) The cells were observed and counted under a light microscope, wherein the cells expressing senescence-specific β-galactosidase were positive cells and showed blue color.

### Example 9 ELISA Assay of Telomerase Reverse Transcriptase

(1) Pre-detection preparation: A standard curve was calculated and plotted using a standard concentration gradient of 400 ng/mL, 200 ng/ml, 100 ng/ml, 50 ng/mL, 25 ng/ml, 12.5 ng/mL, 6.25 ng/mL, and 0 ng/mL.
(2) 100 μL of the standards and samples were added to each well of a 96-well plate. The plate was covered with a provided sealing film, and incubated at 37° C. for 2 hours. The name of the sample was noted on the surface of the sealing film.
(3) The sample in each well was discarded and spin-dried without washing.
(4) 100 μL of Biotin-antibody (1×) was added to each well. The plate was covered with a sealing film, and incubated at 7° C. for 1 hour. If Biotin-antibody (1×) appeared cloudy, Biotin-antibody was warmed to room temperature and stirred gently until clear.
(5) The sample in each well was discarded. Each well was added with 200 μL of Wash Buffer using a multichannel pipette and left to stand for 2 minutes for washing. This process was repeated twice for a total of three times of washing. Complete removal of liquid at each step was critical for good performance. After the last time of washing, the Wash Buffer was discarded, and the 96-well plate was turned upside down and dried with a clean paper.
(6) 100 μL of HRP-avidin (1×) was added to each well. The 96-well microtiter plate was covered with a new sealing film and incubated at 37° C. for 1 hour.
(7) The process of discarding samples and washing in step 6 was repeated 5 times.
(8) 90 μL of TMB Substrate was added to each well. The plate was incubated in the dark at 37° C. for 15-30 minutes. 50 μL of Stop Solution was added to each well, and the plate was gently taped to mix solution evenly.
(9) The optical density of each well was detected at 450 nm within 5 min using a microtiter plate reader.

### Example 10 Detection of the Binding of Polypeptide Microarray to Recombinant Protein

(1) Synthesis of polypeptide chip: A polypeptide chip was designed by overlapping and synthesized according to the sequence of KDM6B protein, with a total of two arrays.
(2) Synthesis of polypeptide microarray: A matrix membrane was activated, and placed on a fully automated polypeptide chip synthesizer. A Fmoc-amino acid solution was automatically transferred to a specific position on the activated membrane according to the set procedure, to react with the membrane. The membrane was immersed sequentially into the blocking solution I and II to block side chains, and washed with dimethyl formamide (DMF). The membrane was placed in a deprotection solution to remove the Fmoc protective group at the amino-terminal, then washed with DMF, and dried with ethanol. The above steps were repeated until all polypeptide microarrays were completely synthesized. After the synthesis was completed, the protective groups of the side chains were removed with specific organic reagents. The membrane was washed with CH2Cl2, and dried with ethanol for immediate use or storage at −20° C.
(3) Blocking: After activated, the polypeptide microarray chips were added with a blocking solution, shaken at room temperature for 4 hours, and washed.
(4) Labeling of target protein with biotin: 1 mL of protein sample of WDR5 synthetic protein with a concentration of 1.5 mg/ml was labeled using EZ-link NHS-PEO4-Biotinylation kit (prod #21455).
(5) Incubation of labeled protein samples with the polypeptide microarray chip: 5 ml of the biotin-labeled WDR5 synthetic protein sample with a final concentration of 1 μg/ml that was diluted with the blocking solution was mixed with the polypeptide microarray chip, and incubated at 4° C. overnight. The control group was incubated with the blocking solution.
(6) Streptavidin-HRP incubation: The reaction reagent Streptavidin-HRP (High Sensitivity Streptavidin-HRP (prod #21133)) was diluted with the blocking solution in an ratio of 1:10000, and then the polypeptide microarray chip was incubated with 5 ml of the diluted Streptavidin-HRP under shaking at room temperature for 2 hours, and washed.
(7) Color development: ECL luminescence reagent was added, and digital imaging was performed on a Chempchemi digital imager.
(8) Scan of polypeptide microarray chip and data analysis of color spot: The colored polypeptide microarray chip was imaged at 425 nm using the Chempchemi Imaging System with 200 s exposure. The optical density of colored spots was analyzed using TotalLab image analysis software, and the optical density of each colored spot was calculated using the “Spot Edge Average” algorithm in the software, with the optical density of the surrounding background of each colored spot as a reference.

### Effect Example 1 Study on the Effect and Regulatory Mechanism of KDM6B/WDR5 on Senescence and Osteogenic Differentiation of Bone Marrow Mesenchymal Stem Cells

Study on the effect and regulatory mechanism of KDM6B on senescence of bone marrow mesenchymal stem cells
1.2 WDR5 could form a protein complex with KDM6B and MLL1 in bone marrow mesenchymal stem cells.

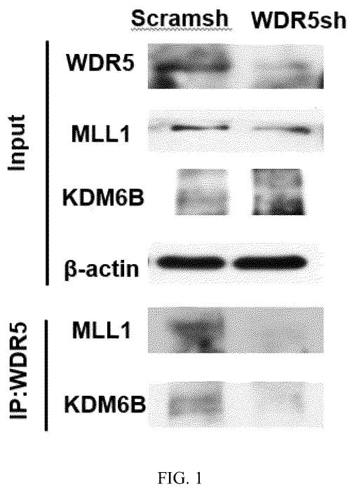

In order to clarify whether WDR5 can form a protein complex with KDM6B and MLL1, bone marrow mesenchymal stem cells with stable knockout of WDR5 were subjected to Co-IP assay. The results of Co-IP assay show that, compared to the control group, in the WDR5 knockout group, the formation of protein complexes of WDR5 with KDM6B and MLL1 was reduced in the bone marrow mesenchymal stem cells (FIG. 1).
1.3 Binding of WDR5 to KDM6B and MLL1 was significantly increased in bone marrow mesenchymal stem cells of senescent mice

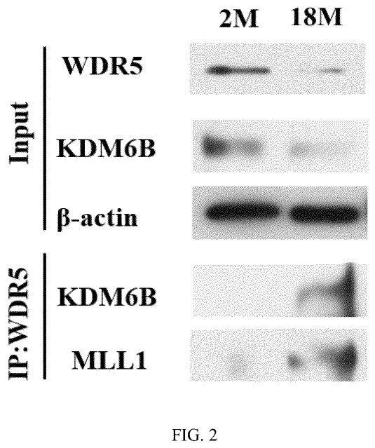

To clarify the regulatory effects of KDM6B, WDR5, and MLL1 on mesenchymal stem cells under senescent conditions, 2 and 18-month-old C57BL/6 mice were subjected to Co-IP assay to detect protein complex formation. The results of Co-IP assay showed that the complex formation of WDR5, KDM6B, and MLL1 was increased in bone marrow mesenchymal stem cells of aged C57BL/6 mice compared to control group (FIGS. 1-2).

### Effect Example 2 Study on the Effect and Regulatory Mechanisms of KDM6B/WDR5 Protein Complex on the Osteogenic Differentiation of Bone Marrow Mesenchymal Stem Cells

3.1 There were 7 binding sites within KDM6B/WDR5, and peptides 102 and 114 could effectively block the formation of the KDM6B/WDR5 protein complex.

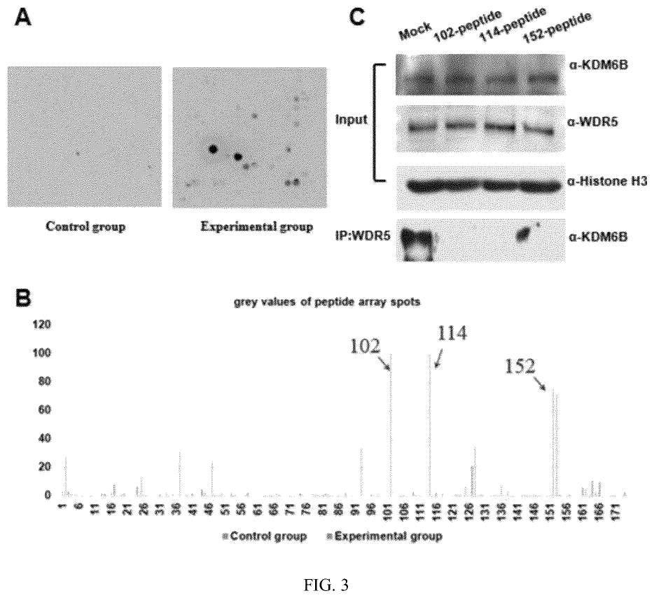

To deeply investigate the functional regulation mechanism of KDM6B on bone marrow mesenchymal stem cells, the polypeptide microarray technology was used to discover the sequence of the binding sites of KDM6B/WDR5 protein complex. It was found by polypeptide microarray technology that there were seven positive binding sites of WDR5 and KDM6B (FIG. 3A). The positive binding sites were selected according to a spot with an optical density value of more than 30% and an optical density value on the negative reaction membrane of less than 30%. According to the analysis on the grey scale of hybridization of the chips, it was found that 102, 114 and 152 showed a higher binding degree among the seven binding sites (FIG. 3B). According to the binding site analysis and sequence design, peptide 102, peptide 114, and peptide 152 were synthesized.

| | Peptide 102: |
|---|---|
| (SEQ ID No. 1) | |
| | FITC-(Acp)-KEKSRRVLGNLDLQSYGRKKRRQRRR; |
| | Peptide 114: |
| (SEQ ID No. 2) | |
| | FITC-(Acp)-ADLTISHCAADVVRAYGRKKRRQRRR; |
| | Peptide_152: |
| (SEQ ID NO. 6) | |
| | FITC-(Acp)-IVPMIHVSWNVARTVYGRKKRRQRRR. |

As detected by Co-IP assay, among the three bioactive polypeptides 102, 114 and 152, only peptide 102 and peptide 114 could effectively open the binding of KDM6B/WDR5 protein complex (FIG. 3C).
3.2 Polypeptides 102 and 114 could inhibit senescence of bone marrow mesenchymal stem cells and promote osteogenic differentiation of bone marrow mesenchymal stem cells

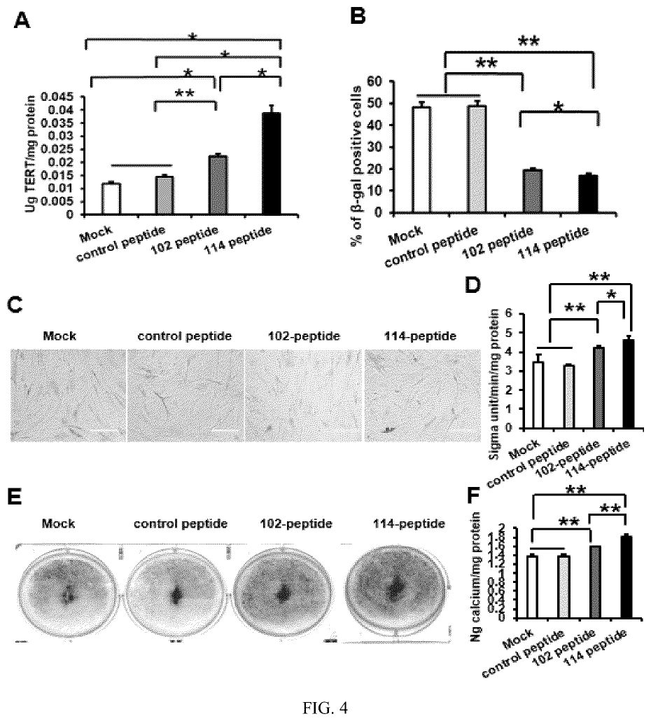

The functional effects of polypeptides 102 and 114 on bone marrow mesenchymal stem cells were simultaneously investigated. The results of ELISA assay of telomerase reverse transcriptase showed that peptide 114 could promote the expression of telomerase reverse transcriptase in bone marrow mesenchymal stem cells compared with the control group (FIG. 4A). The results of β-gal staining and quantitative analysis show that peptide 102 and peptide 114 led to a significant reduction in the number of β-gal-positive cells in bone marrow mesenchymal stem cells compared to the control group (FIG. 4B, C). After 3 days of induction in osteogenic culture medium, it was found that peptide 102 and peptide 114 promoted ALP activity of bone marrow mesenchymal stem cells compared to the control group (FIG. 4D). Alizarin red staining and calcium quantification were performed after 2 weeks of culture in osteogenic induction culture medium. The results show that the mineralization capacity of bone marrow mesenchymal stem cells in the peptide 102 and peptide 114 groups was significantly higher compared to the control group (FIG. 4E, F).

TABLE 2
Data for FIG. 4A

| | | SD |
|---|---|---|
| Mock | 0.011714 | 0.000928673 |
| control peptide | 0.014349 | 0.000934984 |
| 102 peptide | 0.022363 | 0.000953919 |
| 114 peptide | 0.038878 | 0.002975362 |

TABLE 3
Data for FIG. 4B

| | | SD |
|---|---|---|
| Mock | 48.21898 | 2.309487 |
| control peptide | 48.83721 | 2.325581 |
| 102 peptide | 19.33356 | 0.989755 |
| 114 peptide | 16.75761 | 1.382366 |

TABLE 4
Data for FIG. 4D

| | | |
|---|---|---|
| Mock | 3.435171 | 0.39624 |
| control peptide | 3.262909 | 0.064275 |
| 102-peptide | 4.217977 | 0.07766 |
| 114-peptide | 4.620564 | 0.191912 |

TABLE 5
Data for FIG. 4F

| | | SD |
|---|---|---|
| Mock | 1.367242 | 0.042852 |
| control peptide | 1.368383 | 0.04055 |
| 102 peptide | 1.584158 | 0.015308 |
| 114 peptide | 1.806783 | 0.063106 |

3.3 Polypeptides 102 and 114 could inhibit the senescence of bone marrow mesenchymal stem cells in aged mice and promote their osteogenic differentiation.

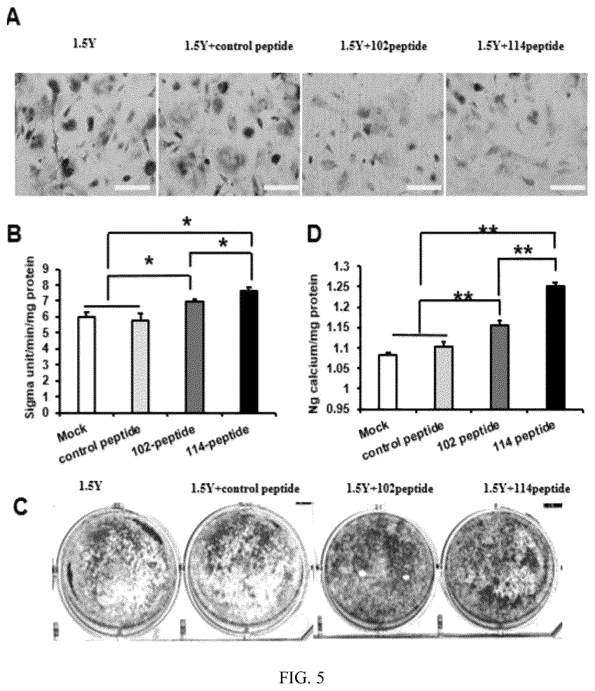

In order to confirm that bioactive polypeptides 102 and 114 can functionally regulate senescent bone marrow mesenchymal stem cells, experiments related to senescence and differentiation functions were carried out. The results of β-gal staining show that peptide 102 and peptide 114 led to a significant reduction in the number of β-gal-positive cells in senescent bone marrow mesenchymal stem cells compared to the control group (FIG. 5A). After 3 days of induction in osteogenic culture medium, it was found that peptide 102 and peptide 114 promoted ALP activity of senescent bone marrow mesenchymal stem cells compared to the control group (FIG. 5B). Alizarin red staining and calcium quantification were performed after 2 weeks of induction in osteogenic culture medium. The results show that the mineralization capacity of senescent bone marrow mesenchymal stem cells in the peptide 102 and peptide 114 groups was significantly higher compared to the control group (FIG. 5C, D).

TABLE 6
Data for FIG. 5B

| | | SD |
|---|---|---|
| Mock | 6.02553 | 0.264389 |
| control peptide | 5.763749 | 0.432205 |
| 102-peptide | 6.950772 | 0.156132 |
| 114-peptide | 7.611731 | 0.242683 |

TABLE 7
Data for FIG. 5D

| | | |
|---|---|---|
| Mock | 1.082396 | 0.006429 |
| control peptide | 1.103677 | 0.011295 |
| 102 peptide | 1.153978 | 0.012147 |
| 114 peptide | 1.251294 | 0.009707 |

### Effect Example 3 Study on the Effect and Regulatory Mechanism of WDR5 on Senescence and Odontogenic Differentiation of Mesenchymal Stem Cells

5.1 KDM6B can form a protein complex with WDR5 in stem cells from apical papilla.

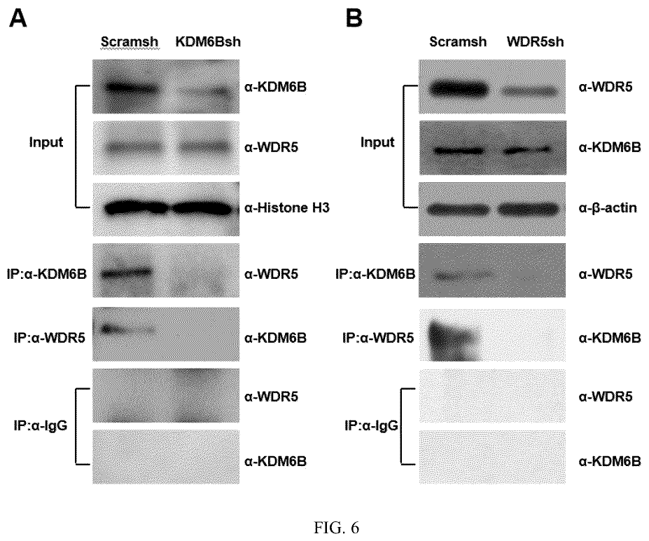

In order to further investigate the regulatory mechanism of KDM6B on stem cells from apical papilla, the binding of KDM6B to WDR5 was detected by Co-IP assay. The results of Co-IP assay show that the binding of KDM6B to WDR5 in the stem cells from apical papilla in the KDM6B knockout group was significantly reduced compared to the control group (FIG. 6A). To further confirm that KDM6B can form a protein complex with WDR5, the stem cells from apical papilla with stable knockout of WDR5 were subjected to Co-IP assay. The results of Co-IP assay show that the formation of the protein complex of KDM6B and WDR5 in the cells in the WDR5 knockout group was reduced compared to the control group (FIG. 6B).

### Effect Example 4 Study on the Effect of KDM6B/WDR5 Protein Complex on the Odontogenic Differentiation of Mesenchymal Stem Cells and its Regulatory Mechanism

7.1 Mutation of the KDM6B sequence at sites 102 and 114 could inhibit senescence of stem cells from apical papilla and promote their odontogenic differentiation.

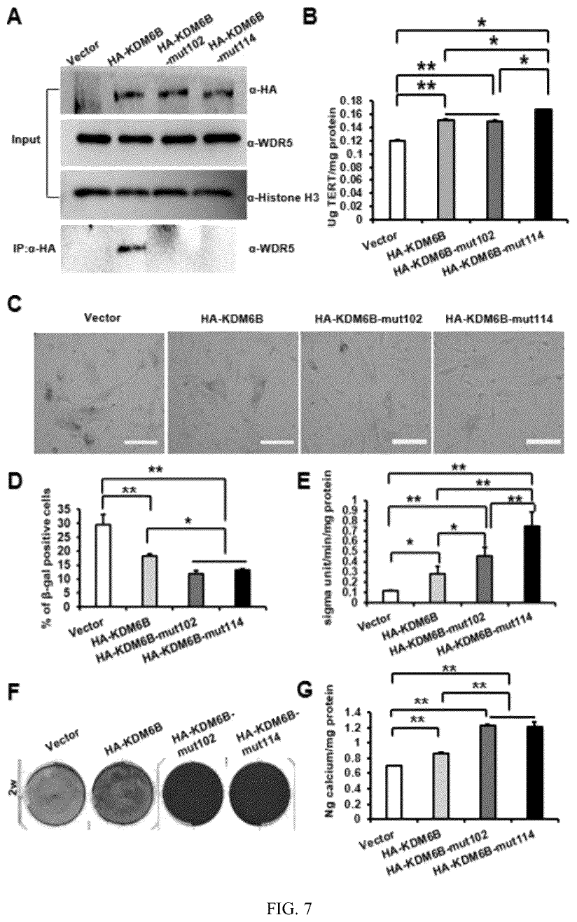

To further determine whether the 102 and 114 sequences could block the formation of the KDM6B/WDR5 protein complex, cells overexpressing KDM6B with mutation at 102 and 114 sites were subjected to Co-IP assay. The experimental results show that the binding of KDM6B to WDR5 in the HA-KDM6B-mut102 group and HA-KDM6B-mut114 group was reduced compared to the HA-KDM6B group (FIG. 7A). Next, the effect of the overexpression of KDM6B with mutation at 102 and 114 sites on senescence of stem cells from apical papilla was detected. The results of ELISA assay of telomerase reverse transcriptase show that the expression of the telomerase reverse transcriptase in stem cells from apical papilla in the HA-KDM6B-mut102 and HA-KDM6B-mut114 groups was promoted compared to the control group (FIG. 7B). The results of β-gal staining and quantitative analysis show that, compared to the control group, the number of β-gal-positive cells in stem cells from apical papilla was significantly reduced in HA-KDM6B-mut102 and HA-KDM6B-mut114 groups (FIG. 7C, D). After 3 days of induction in odontogenic culture medium, it was found that the ALP activity of stem cells from apical papilla in the HA-KDM6B-mut102 and HA-KDM6B-mut114 groups were promoted compared to the control group (FIG. 7E). Alizarin red staining and calcium quantification were performed after 2 weeks of indication in odontogenic culture medium. The results show that the mineralization capacity in the HA-KDM6B-mut102 and HA-KDM6B-mut114 groups was significantly higher than that in the control group (FIG. 7F. G).

TABLE 8
Data for FIG. 7B

| | | SD |
|---|---|---|
| Vector | 0.120301 | 0.002321238 |
| HA-KDM6B | 0.151428 | 0.001219 |
| HA-KDM6B-mut102 | 0.148849 | 0.002428533 |
| HA-KDM6B-mut114 | 0.167124 | 0.001247402 |

TABLE 9
Data for FIG. 7D

| | MEAN | SD |
|---|---|---|
| Vector | 29.44052 | 3.676117107 |
| HA-KDM6B | 18.2746 | 0.752269296 |
| HA-KDM6B-mut102 | 11.74683 | 1.302423965 |
| HA-KDM6B-mut114 | 13.15876 | 0.433142542 |

TABLE 10
Data for FIG. 7E

| | | SD |
|---|---|---|
| Vector | 0.116858 | 0.005575 |
| HA-KDM6B | 0.28432 | 0.069515 |
| HA-KDM6B-mut102 | 0.459554 | 0.082139 |
| HA-KDM6B-mut114 | 0.751555 | 0.133336 |

TABLE 11
Data for FIG. 7G

| | | |
|---|---|---|
| Vector | 0.704162 | 0.004041 |
| HA-KDM6B | 0.865251 | 0.008312 |
| HA-KDM6B-mut102 | 1.235037 | 0.007506 |
| HA-KDM6B-mut114 | 1.212432 | 0.071305 |

7. 2 Polypeptides 102 and 114 could inhibit senescence of stem cells from apical papilla and promote their odontogenic differentiation.

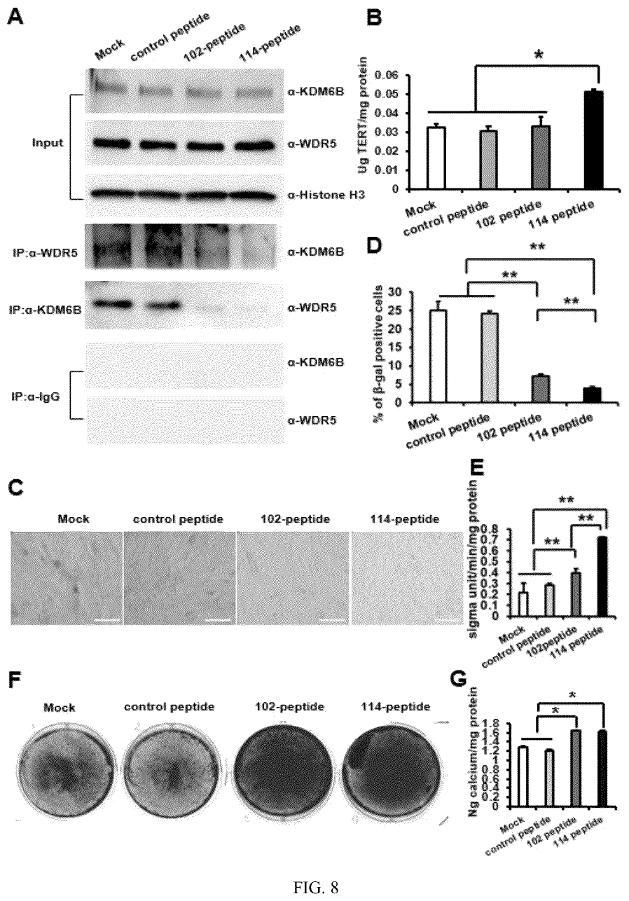

Based on the previous study, the present disclosure develops and utilizes small-molecule polypeptides targeting the binding region of KDM6B to WDR5 to regulate the function of stem cells from apical papilla by regulating the binding of KDM6B/WDR5 complex. After treated with the bioactive polypeptides, the stem cells from apical papilla were subjected to Co-IP assay. The experimental results show that the binding of KDM6B to WDR5 in stem cells from apical papilla in the peptide 102 and peptide 114 groups was reduced compared to the control group (FIG. 8A). Then the effects of peptide 102 and peptide 114 on senescence of stem cells from apical papilla were detected. The results of the ELISA assay of telomerase reverse transcriptase show that peptide 114 promoted the expression of telomerase reverse transcriptase in stem cells from apical papilla compared to the control group (FIG. 8B). The results of β-gal staining and quantitative analysis show that peptide 102 and peptide 114 led to a significant reduction in the number of β-gal-positive cells in stem cells from apical papilla compared to the control group (FIG. 8C, D). After 3 days of induction in odontogenic culture medium, it was found that peptide 102 and peptide 114 promoted ALP activity of the stem cells from apical papilla compared to the control group (FIG. 8E). Alizarin red staining and calcium quantification were performed after 2 weeks of induction in odontogenic culture medium. The results show that the mineralization capacity of the stem cells from apical papilla in the peptide 102 and peptide 114 groups was significantly higher than that in the control group (FIG. 8F, G).
7.3 There was a downstream target gene co-regulated by KDM6B and WDR

TABLE 12
Data for FIG. 8B

| | | SD |
|---|---|---|
| Mock | 0.032608 | 0.001955173 |
| control peptide | 0.030542 | 0.002918559 |
| 102 peptide | 0.033312 | 0.004895822 |
| 114 peptide | 0.051677 | 0.001020189 |

TABLE 13
Data for FIG. 8D

| | | SD |
|---|---|---|
| Mock | 25.07861 | 2.419658 |
| control peptide | 24.20205 | 0.643601 |
| 102 peptide | 7.29624 | 0.448604 |
| 114 peptide | 3.978156 | 0.35091 |

TABLE 14
Data for FIG. 8E

| | | SD |
|---|---|---|
| Mock | 0.218303 | 0.087875 |
| control peptide | 0.285743 | 0.012709 |
| 102peptide | 0.396121 | 0.038258 |
| 114 peptide | 0.723759 | 0.003245 |

TABLE 15
Data for FIG. 8G

| | | |
|---|---|---|
| Mock | 1.28938 | 0.031225 |
| control peptide | 1.209692 | 0.033382 |
| 102 peptide | 1.63736 | 0.016823 |
| 114 peptide | 1.619093 | 0.032005 |

### Effect Example 5

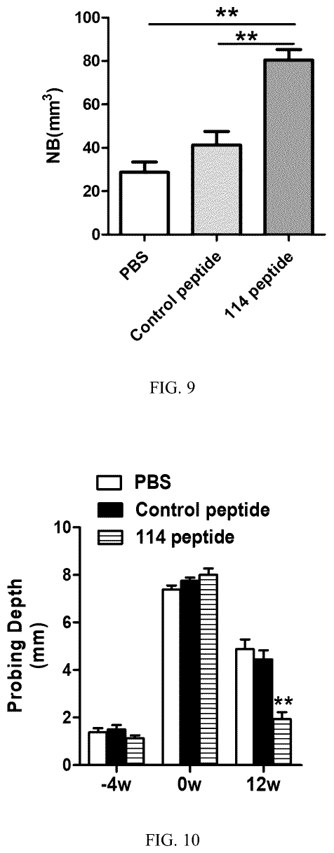

1. The new bone formation volume (mm³) in the minipig periodontitis model is shown in Table 16 and FIG. 9.

TABLE 16
Data for FIG. 9

| PBS | Control peptide | 114 peptide |
|---|---|---|
| 23.73 | 26.69 | 80.68 |
| 48.06 | 69.27 | 79.08 |
| 21.96 | 47.63 | 80.48 |
| 23.9 | 38.3 | 76.48 |
| 16.85 | 32.25 | 85.49 |
| 37.64 | 33.48 | 101.99 |

2. Clinical indicators for minipig periodontitis model
   Establishment of an Experimental Minipig Periodontitis Model with Bone Defect

Method: An experimental minipig periodontitis model was established by creating bone defect and ligating silk threads. Specifically, minipigs were conventionally anaesthetized, and extraorally disinfected. The mandibular first permanent molar of the minipig was selected as the experimental tooth, which was subjected to intra-sulcal incision and vertical incision. The mucoperiosteal flap was opened, the mesia-buccal root of the mandibular first permanent molar of the minipig was exposed by bone removal, and a bone defect with a size of 3 mm×5 mm×7 mm was created. That is, 3 mm×5 mm×7 mm of the mesial proximal alveolar bone was removed, and the mesiobuccal root buccal bone plate and distal alveolar bone (3 mm×5 mm, the gingival depth was level with the mesial proximal surface) were removed. The suture in situ was performed. The experimental tooth neck was ligated with silk threads. The bone defect was not able to be self-repaired within 4 weeks after surgery, indicating that a periodontal bone defect model was successfully established. The successful establishment of periodontal bone defect model was verified by CT imaging and clinical examination.

Regeneration and Repair by Bioactive Polypeptide 114 in Minipigs with Experimental Periodontal Bone Defect

12 Wuzhishan minipigs were used to establish periodontitis model, with a total of 24 sides, which were randomly divided into 3 groups. Each side was injected with a drug at an amount of 60 μl. Group 1: flap surgery with scraping+injection of sterile saline after modeling (untreated control group); group 2: flap surgery with scraping+injection of control polypeptide after modeling (control group); group 3: flap surgery with scraping+injection of bioactive polypeptide 114 after modeling (experimental group).

Drug injection treatment was performed 4 weeks after the modeling operation. The effect of bioactive polypeptide 114 on the regeneration and repair capacity of defected tissues mediated by stem cells in minipigs of periodontitis was assessed by comparing the clinical indicators and imaging examination after modeling (−4 w), 4 weeks after modeling without drug injection (0 w) and 3 months after drug treatment (12 w).

The results of CT imaging, Geomagic Studio 12 analysis, mimics medical 17 analysis of bone volume, and clinical examination show that the bioactive polypeptide 114 promoted the mesenchymal stem cells-mediated regeneration of periodontal tissue.

### Post-Treatment Observation

### Observation of Clinical, Imaging and Histological Indicators

Clinical indicators (PD, AL and GR) detection and CT imaging were performed before the experiment, after modeling (−4 w) and 3 months after treatment (12 w).

Periodontal probing depth (PD): The depth of the periodontal pocket on the mesiobuccal side of the tooth was recorded at a probing pressure of 20-25 g.

Gingival recession (GR): The distance from the cemento-enamel junction to the gingival margin was measured using a periodontal probe. If there was gingival recession, the cementoenamel junction was exposed, and the gingival margin was located at the root of the cementoenamel junction, then the distance between the two was recorded as a positive value; if there was no gingival recession, the gingival margin was located at the crown of the cementoenamel junction, then the distance between the two was recorded as a negative value.

Attachment loss (AL): The degree of attachment loss was determined by subtracting GR from the periodontal pocket depth. If the two numbers were subtracted to zero, or the cementoenamel junction cannot be detected, it means there was no attachment loss. If the gingival recession caused the gingival margin to locate at the root of the cementoenamel junction, the two numbers were added to obtain the degree of the attachment loss.
2.1 Probing depth (PD) was shown in Table 17 and FIG. 10.

TABLE 17
Data for FIG. 10

| | PBS | | | | | | | | Control peptide | | | | | | | | 114 peptide | | | | | | | |
|---|---|---|---|---|---|---|---|---|---|---|---|---|---|---|---|---|---|---|---|---|---|---|---|---|
| −4 w | 1 | 1 | 1 | 2 | 1 | 2 | 2 | 1 | 1 | 1 | 2 | 2 | 2 | 1 | — | — | 1 | 1 | 1 | 1 | 1 | 1 | 2 | 1 |
| 0 w | 8 | 7 | 8 | 7 | 8 | 7 | 7 | 7 | 8 | 8 | 7 | 8 | 7.5 | 7.5 | — | — | 7.5 | 8.5 | 7.5 | 7 | 8 | 9 | 9 | 7.5 |
| 12 w | 3 | 4.5 | 5.5 | 7 | 5 | 4 | 5 | 5 | 6 | 4 | 4 | 4 | 3 | 3.5 | — | — | 2.5 | 2 | 2 | 0.5 | 1 | 2 | 2.5 | 3 |

wherein, ‘—’ represents an absence of data due to the death of an experimental animal.

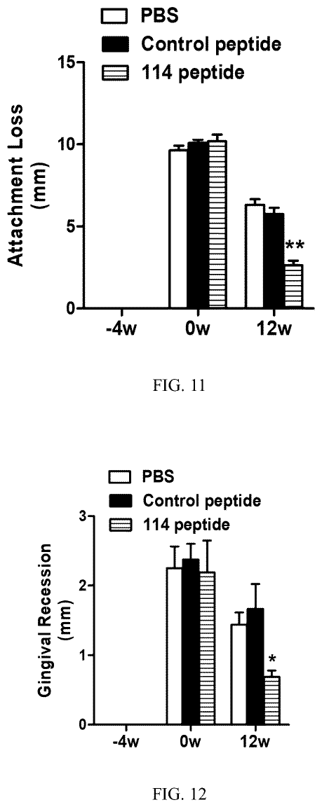

12 weeks after injection of bioactive polypeptide in minipig periodontitis model, it was found that the probing depth in the 114 peptide group was significantly lower than that in the PBS group and the control peptide group, as measured by using a periodontal probe. The difference between the 114 peptide group and the other groups was significant; *P≤0.05.
2. 2 Attachment loss (AL) is shown in Table 18 and FIG. 11.

TABLE 18
Data for FIG. 11

| | PBS | | | | | | | | Control peptide | | | | | | | 114 peptide | | | | | | |
|---|---|---|---|---|---|---|---|---|---|---|---|---|---|---|---|---|---|---|---|---|---|---|
| −4 w | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 0 w | 10 | 9.5 | 10 | 9.5 | 9.5 | 9.5 | 8 | 11 | 10 | 10 | 10 | 9.5 | 10 | 11 | 10 | 9.5 | 10 | 9.5 | 9 | 9.5 | 12 | 12 |
| 12 w | 4.5 | 6.5 | 7 | 7.5 | 6 | 5.5 | 6.5 | 7 | 6.5 | 7 | 6 | 5 | 4.5 | 5.5 | 3 | 2.5 | 2.5 | 1.5 | 1.5 | 3 | 3 | 4 |

2. 3 Gingival Recession (GR) is shown in Table 19 and FIG. 12.

12 weeks after injection of bioactive polypeptide in minipig periodontitis model, it was found that the attachment loss level in the 114 peptide group was significantly lower than that in the PBS group and the control peptide group, as measured by using a periodontal probe. The difference between the 114 peptide group and the other groups was significant; *P≤0.05.

TABLE 19
Data for FIG. 12

| | PBS | | | | | | | | Control peptide | | | | | | | | 114 peptide | | | | | | | |
|---|---|---|---|---|---|---|---|---|---|---|---|---|---|---|---|---|---|---|---|---|---|---|---|---|
| −4 w | 0 | 0 | 0 | 0 | 0 | 0 | 0 | 0 | 0 | 0 | 0 | 0 | 0 | 0 | — | — | 0 | 0 | 0 | 0 | 0 | 0 | 0 | 0 |
| 0 w | 2 | 2.5 | 2 | 2.5 | 1.5 | 2.5 | 1 | 4 | 2 | 2 | 3 | 1.5 | 2.5 | 3.5 | — | — | 2.5 | 1 | 2.5 | 2.5 | 1 | 0.5 | 3 | 4.5 |
| 12 w | 1.5 | 2 | 1.5 | 0.5 | 1 | 1.5 | 1.5 | 2 | 0.5 | 3 | 2 | 1 | 1.5 | 2 | — | — | 0.5 | 0.5 | 0.5 | 1 | 0.5 | 1 | 0.5 | 1 |

wherein, ‘—’ represents an absence of data due to the death of an experimental animal.

12 weeks after injection of bioactive polypeptide in minipig model of periodontitis, it was found that the degree of gingival recession in the 114 peptide group was significantly lower than that in the PBS group and the control peptide group, as measured by using a periodontal probe. The difference between the 114 peptide group and the other groups was significant; *P≤0.05.

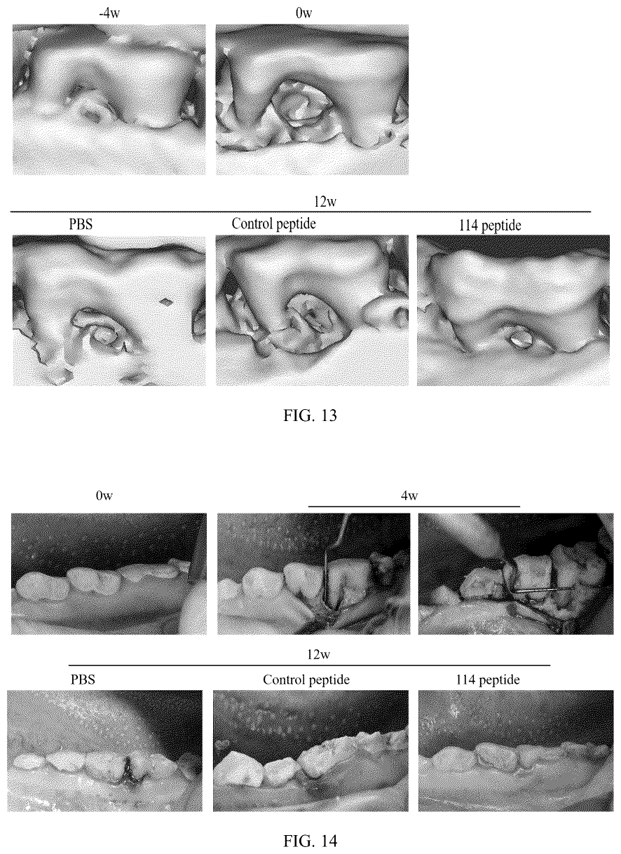

As shown in FIG. 13, the natural occlusal position of the tested minipigs lying flat on the imaging table of the CBCT scanner with the head fixed was continuously scanned to obtain CBCT tomographic images, and the DICOM images was subjected to three-dimensional reconstruction by using Mimics 17.0 image processing software. The CBCT data was obtained before, after modeling and after surgery for post-treatment evaluation. The postoperative three-dimensional reconstruction model showed that there was new bone formation in the PBS group, control peptide group and 114 peptide group; and the effect of new bone formation in the 114 peptide group was better than that in the control peptide group and the PBS group.

FIG. 14 shows the intraoral images. At week 12 after injection of the bioactive polypeptide, wounds in the bone defect area of periodontal tissues of minipigs in the PBS group, control peptide group and 114 peptide group exhibited healing, no infected and necrotic tissues were seen; and the restoration effect of soft tissues in the 114 peptide group was better than that in the control peptide group and the PBS blank control group.

### Effect Example 6

### 1. Palatal Mucosa Defect

Fifteen 8-month-old male Balb/c mice were randomly divided into three groups: PBS group, control group and 114 group, with five mice in each group.

Before the establishment of the defect model, the mice were anaesthetized by injection of 4% chloral hydrate at 0.2 ml/20 g, and the skin was prepared. A circular full-thickness skin defect with a diameter of 6 mm on the back of the mice was created using a skin punch. On the day of the defect creation, the third day, the seventh day, the tenth day, the fourteenth day, the seventeenth day, and the twenty-first day after the defect creation, an equal amount of 100 μl of PBS, 10 μl/ml control peptide, and 10 μl/ml 114 peptide was separately injected at four equidistant sites at 2 mm from the edge of the defect.

The defect region was photographed before each injection, and the area of the defect region was calculated by using Image-Pro plus software. The healing rate was calculated as: healing rate=(area of the defect region on day 0−area of the defect region on day 14/21)/area of the defect region on day 0. T-test was performed and showed p<0.05, indicating that the healing rate of skin defect on days 14 and 21 was statistically significant compared to the control group.

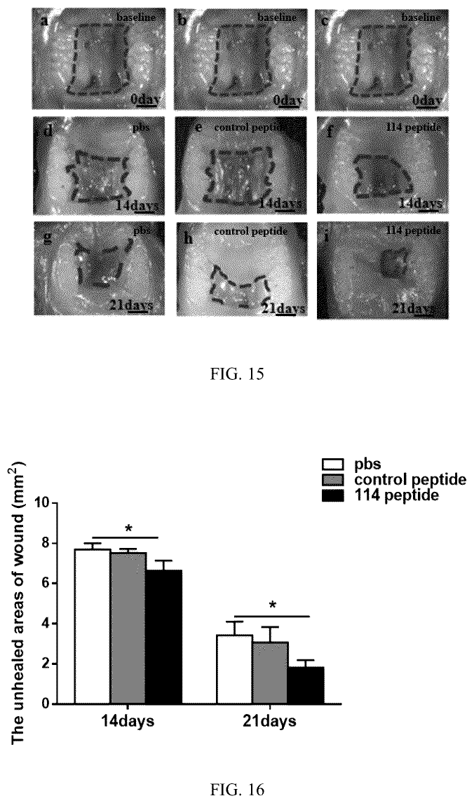

The results are shown in FIGS. 15-16 and Table 20.

TABLE 20
Data for FIG. 16

| 14-PBS | 14-control | 14-114 | 21-PBS | 21-control | 21-114 |
|---|---|---|---|---|---|
| 8.553957 | 8.083566 | 5.781627 | 4.259305 | 2.598592 | 2.259207 |
| 7.139076 | 7.187624 | 7.253595 | 2.066862 | 2.159928 | 0.3319243 |
| 7.393813 | 7.306628 | 8.230819 | 3.930576 | 2.134038 | 1.963786 |
| 7.693449 | 7.503132 | 5.520400 | 3.418914 | 5.362377 | 2.329762 |
| 7.695074 | 7.520237 | 6.400598 | 3.418914 | 3.063734 | 2.213450 |

The analysis of the area of unhealed defected palatal mucosa of mice in the PBS, control peptide, and 114 peptide groups on days 14 and 21 showed a statistical significance in the 114 peptide group compared to the PBS and control peptide groups; *P≤0.05.

### 2. Skin Defect

Fifteen 8-month-old male Balb/c mice were randomly divided into three groups: PBS group, control group and 114 group, with five mice in each group.

Before the establishment of the defect model, the mice were anaesthetized by injection of 4% chloral hydrate at 0.2 ml/20 g, and the skin was prepared. A circular full-thickness skin defect with a diameter of 6 mm on the back of the mice was created using a skin punch. On the day of the defect creation, the third day, the seventh day, the tenth day, the fourteenth day, the seventeenth day, and the twenty-first day after the defect creation, an equal amount of 100 μl of PBS, 10 μl/ml control peptide, and 10 μl/ml 114 peptide was separately injected at four equidistant sites at 2 mm from the edge of the defect.

The defect region was photographed before each injection, and the area of the defect region was calculated by using Image-Pro plus software. The healing rate was calculated as: healing rate=(area of the defect region on day 0-area of the defect region on day 14/21)/area of the defect region on day 0. T-test was performed and showed p<0.05, indicating that the healing rate of skin defect on days 14 and 21 was statistically significant compared to the control group.

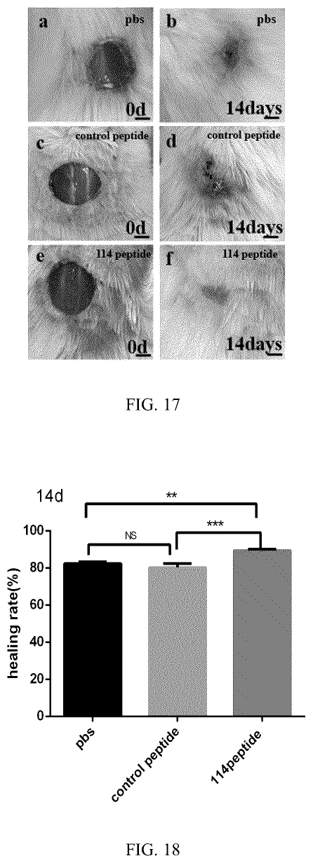

The results are shown in FIGS. 17-18 and Table 21.

TABLE 21
Data for FIG. 18

| Group/Number | | | | Healing rate | |
|---|---|---|---|---|---|
| Control | Day 0 | Day 10 | Day 14 | Day 10 | Day 14 |
| 1 | 4135 | 3936 | 882 | 0.048126 | 0.786699 |
| 2 | 7204 | 3158 | 1734 | 0.561632 | 0.7593 |
| 3 | 4942 | 1415 | 639 | 0.713679 | 0.8707 |
| 4 | 5709 | 1259 | 991 | 0.779471 | 0.826414 |
| 5 | 5053 | 2410 | 1036 | 0.523056 | 0.794973 |
| PBS | | | | | |
| 1 | 6949 | 1911 | 1002 | 0.724996 | 0.855807 |
| 2 | 6943 | 5897 | 1151 | 0.150655 | 0.834222 |
| 3 | 5022 | 2115 | 885 | 0.578853 | 0.823775 |
| 4 | 5714 | 5056 | 1070 | 0.115156 | 0.812741 |
| 5 | 6120 | 3935 | 1132 | 0.357026 | 0.815033 |
| 114 | | | | | |
| 1 | 6226 | 1396 | 681 | 0.775779 | 0.89062 |
| 2 | 6554 | 3701 | 722 | 0.435307 | 0.889838 |
| 3 | 6917 | 2313 | 681 | 0.665606 | 0.901547 |
| 4 | 5532 | 983 | 486 | 0.822307 | 0.912148 |
| 5 | 4700 | 1816 | 715 | 0.613617 | 0.847872 |

The analysis of the area of unhealed defected palatal mucosa of mice in the PBS, control peptide, and 114 peptide groups on days 10 and 14 showed a statistical significance in the 114 peptide group compared to the PBS and control peptide groups; *P≤0.05, **P≤0.01, ***P≤0.001.

### 3. Prevention of Bone Loss in OVX Mouse of Osteoporosis Model

Twenty five 3-month-old C57BL/6 mice were purchased from Vital River and randomly divided into sham operation group, OVX group, PBS group, control peptide group and 114 peptide group, with five mice in each group. The osteoporosis model was established by removing bilateral ovaries in OVX, PBS, control peptide and 114 peptide groups. In the sham operation group, ovaries were not removed. 6 weeks after ovary removal, the mice were intraperitoneally injected with control peptide and peptide 114 at 10 mg/kg for continuous 3 months, and then sacrificed. For the distal femur, the left distal femur of each mouse was scanned ex vivo using a micro-CT system. Each segment of bone trabeculae was selected for segmentation, and subjected to 3D reconstruction. BMD was calculated.

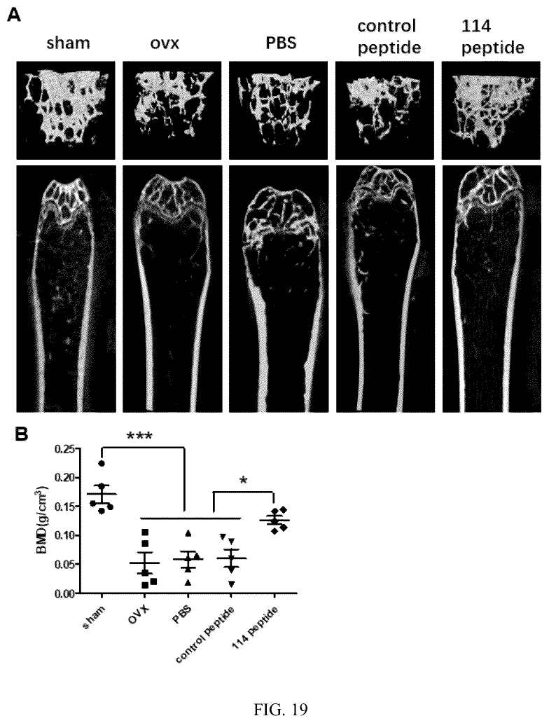

The results are shown in FIG. 19 and Table 22.

TABLE 22
Data for FIG. 19B.

| sham | OVX | PBS | control peptide | 114 peptide |
|---|---|---|---|---|
| 0.1551 | 0.1060 | 0.0191 | 0.0158 | 0.1440 |
| 0.1848 | 0.0355 | 0.0612 | 0.0600 | 0.1242 |
| 0.1490 | 0.0863 | 0.1043 | 0.0894 | 0.1416 |
| 0.2242 | 0.0203 | 0.0659 | 0.0978 | 0.1071 |
| 0.1421 | 0.0143 | 0.0415 | 0.0400 | 0.1137 |

The metaphysis of the mouse femur was subjected to micro-CT analysis. It was found that there was no significant difference in bone density of femoral bone trabeculae of the mice injected with PBS and control peptide compared to the mice injected with OVX. The bone density of femoral bone trabeculae in OVX group, PBS group and control peptide group showed a significant reduction compared to sham group. The mice in 114 peptide group showed a significant up-regulation of bone density of femoral bone trabeculae compared to OVX group, PBS group and control peptide group.

The above provides a detailed description of the polypeptide sequences of KDM6B provided by the present disclosure and use thereof in the regulation of mesenchymal stem cell function. The principles and embodiments of the present disclosure have been described with reference to specific examples, and the description of the above embodiments is only to assist in understanding the method of the present disclosure and the core idea thereof. It should be noted that, several improvements and modifications may be made to the present disclosure by those skilled in the art without departing from the principle of the present disclosure, and the improvements and modifications shall fall within the protection scope of the claims.

1. A method of inhibiting senescence of mesenchymal stem cells, promoting osteogenic differentiation or odontogenic differentiation of mesenchymal stem cells, comprising administering an agent or a medicament targeting a protein complex to a subject in need thereof;
wherein the protein complex is selected from the group consisting of a first protein complex and a second protein complex;
wherein, the first protein complex comprises WDR5 and KDM6B, and
the second protein complex comprises KDM6B, WDR5 and MLL1.

2. A bioactive peptide, comprising:
(I) an amino acid sequence set forth in SEQ ID No.: 1 or 2,
(II) an amino acid sequence derived from the amino acid sequence set forth in (I) by substitution, deletion or addition of one or more amino acids and having the same functions as the amino acid sequence set forth in (I), or
(III) an amino acid sequence having more than 90% identity to the amino acid sequence set forth in (I) or (II).

3. A method of inhibiting senescence of mesenchymal stem cells,
promoting osteogenic differentiation of bone marrow mesenchymal stem cells and/or promoting odontogenic differentiation of stem cells from apical papilla, comprising administering the bioactive peptide according to claim 2 to a subject in need thereof.

4. A method of preventing and treating osteoporosis and periodontitis, and/or for repairing damaged mucosa and skin, comprising administering the bioactive peptide according to claim 2 to a subject in need thereof.

5. An agent or a medicament comprising the bioactive peptide according to claim 2 and a pharmaceutically acceptable excipient.


SEQUENCE LISTING (15 sequences)

SEQ ID NO: 1 - protein, 26 aa, Artificial Sequence
  Synthetic polypeptide
KEKSRRVLGNLDLQSYGRKKRRQRRR

SEQ ID NO: 2 - protein, 26 aa, Artificial Sequence
  Synthetic polypeptide
ADLTISHCAADVVRAYGRKKRRQRRR

SEQ ID NO: 3 - DNA, 22 bp, Artificial Sequence
  Synthetic polynucleotide
cggaccaatacgaccaaatccg

SEQ ID NO: 4 - DNA, 20 bp, Artificial Sequence
  Synthetic polynucleotide
agccacatcgctcagacacc

SEQ ID NO: 5 - DNA, 28 bp, Artificial Sequence
  Synthetic polynucleotide
cgtggacaaagaagatagcaactccacg

SEQ ID NO: 6 - DNA, 24 bp, Artificial Sequence
  Synthetic polynucleotide
ttccggctctctatctcaatgttt

SEQ ID NO: 7 - DNA, 20 bp, Artificial Sequence
  Synthetic polynucleotide
caaggtaagcctgcagtggc

SEQ ID NO: 8 - DNA, 20 bp, Artificial Sequence
  Synthetic polynucleotide
actgccaatctcatggtcgt

SEQ ID NO: 9 - DNA, 26 bp, Artificial Sequence
  Synthetic polynucleotide
cgacataggtcacaatgaggatgtcg

SEQ ID NO: 10 - DNA, 22 bp, Artificial Sequence
  Synthetic polynucleotide
ttgcttccagctacttgaggtc

SEQ ID NO: 11 - protein, 15 aa, Artificial Sequence
  Synthetic polypeptide
RESRVQRSRMDSSVS

SEQ ID NO: 12 - protein, 15 aa, Artificial Sequence
  Synthetic polypeptide
CETLVERVGRSATDP

SEQ ID NO: 13 - protein, 15 aa, Artificial Sequence
  Synthetic polypeptide
SRSHTTIAKYAQYQA

SEQ ID NO: 14 - protein, 26 aa, Artificial Sequence
  Synthetic polypeptide
IVPMIHVSWNVARTVYGRKKRRQRRR

SEQ ID NO: 15 - protein, 15 aa, Artificial Sequence
  Synthetic polypeptide
VARTVKISDPDLFKM